(19) United States
(12) Patent Application Publication
(10) Pub. No.: US 2023/0316054 A1
(43) Pub. Date: Oct. 5, 2023

(54) MACHINE LEARNING MODELING OF PROBE INTENSITY

(71) Applicant: Illumina Software, Inc., San Diego, CA (US)

(72) Inventors: Yong Li, San Diego, CA (US); Jennifer Zou, San Diego, CA (US)

(73) Assignee: Illumina Software, Inc., San Diego, CA (US)

(21) Appl. No.: 18/129,565

(22) Filed: Mar. 31, 2023

Related U.S. Application Data
(60) Provisional application No. 63/326,226, filed on Mar. 31, 2022.

(51) Int. Cl. G06N 3/08; G16B 20/00

(52) U.S. Cl. CPC G06N 3/0499; G16B 20/00; G06N 3/0464

(57) ABSTRACT

Systems, methods, and apparatus are described herein for training machine learning models to predict probe intensity values using sample-specific image data and/or applying the predicted probe intensity values. As described herein, sample-specific image may include a signal associated with a sample for a process probe in a microarray relating to a single individual. The machine learning model may be trained, using the sample-specific image data, to predict a probe intensity value. The probe intensity value may be a raw probe intensity value or a normalized probe intensity value. After being trained, the machine learning model may receive as input a probe sequence or probe features. The machine learning model may be used to predict a total probe intensity value based on the probe sequence or the one or more probe features.

### CROSS-REFERENCE TO RELATED APPLICATIONS

This application claims the benefit of U.S. Provisional Patent Application No. 63/326,226, filed Mar. 31, 2022, which is incorporated by reference herein in its entirety.

### BACKGROUND

High-throughput genomic technology has revolutionized the landscape of biomedical research. One of the early representative high-throughput genomic technologies is the microarray, which has been a dominant technology for methylation quantification and genotyping. Genotyping microarrays are also referred to as single-nucleotide polymorphism (SNP) arrays, and have been the tool of choice for genome-wide association studies (GWASs) for many years.

### SUMMARY

Systems, methods, and apparatus are described herein for training machine learning models to predict probe intensity values using sample-specific image data and/or applying the predicted probe intensity values. As described herein, sample-specific image data may be received from a genotyping device. The sample-specific image data may include a signal associated with a sample for a probe in a microarray relating to a single individual. The microarray may include a BeadArray, for example. The sample-specific image data may include a raw x signal having a first intensity value of a first colored signal that represents a fluorescent label for a genotype A and a raw y signal having a second intensity value of a second colored signal that represents a fluorescent label for a genotype B. An observed probe intensity value may be identified for the sample based on the sample-specific image data.

The machine learning model may be trained, using the sample-specific image data, to determine a predicted probe intensity value. The training may be based on an input of a probe sequence or probe features. The probe sequence may include an entire probe sequence or a portion thereof. For example, the probe sequence may include a variety of lengths within the entire probe sequence or the entire probe sequence. The predicted probe intensity value may be a predicted total signal intensity of the signal associated with the sample for the probe.

The probe intensity value may be a raw probe intensity value or a normalized probe intensity value. When the probe intensity value is a raw probe intensity value, the predicted probe intensity value may be a predicted raw probe intensity value. When the probe intensity value is a normalized probe intensity value, the predicted probe intensity value may be a predicted normalized probe intensity value. The normalized probe intensity value may be calculated as the sum of the normalized x and y intensities, the Euclidean norm of the normalized x and y intensities, or a Log R ratio.

The machine learning model may be a linear regression model, a random forest model, or a neural network. The machine learning model may receive as input the one or more probe features. The probe features may include probe sequence features (e.g., kmers, entropy, and/or one-hot encoding) and/or genomic context features (e.g., other features). Though genomic context features may be described, these probe features may also be referred to as annotation features, as these features may be derived from external annotations of the genome/epigenome. The machine learning model receives as input the one or more probe features as an entire predefined set of probe features.

The neural network may be a hybrid neural network comprising a convolutional portion and a fully-connected feed forward portion. The input to the neural network may comprises a probe sequence (e.g., 50 bp) for the convolutional portion and one or more probe features for the fully-connected feed forward portion.

After being trained, the machine learning model may receive as input at least one of the probe sequence or the one or more probe features in test data. The machine learning model may be used to predict a total probe intensity value based on the probe sequence or the one or more probe features. The predicted total probe intensity value may include a predicted raw probe intensity value or a predicted normalized probe intensity value.

The predicted total probe intensity value may be applied. For example, when the predicted total probe intensity value comprises a predicted raw probe intensity value, the predicted raw probe intensity value may be applied for background and gradient removal in a region of sample-specific image data received from a genotyping device. When the predicted total probe intensity value comprises a predicted normalized probe intensity value, the predicted normalized probe intensity value may be applied by replacing an expected normalized probe intensity that is used to calculate a Log R ratio value for call number variant (CNV) calling. In another example, the predicted total probe intensity value may be applied to indicate a quality level of the probe.

### BRIEF DESCRIPTION OF THE DRAWINGS

FIG. 1 illustrates a schematic diagram of a system environment.

FIG. 2A is an illustration indicating probes that may be positioned on an image-generating chip in a genotyping device.

FIG. 2B illustrates a system that may be implemented for imaging the probes and processing the image signals for genotype calling based on the colored signals detected from the fluorescent labels.

FIG. 3 includes a graph illustrating clusters of data based on data from a two-channel microarray platform transformed to polar coordinates.

FIG. 4 includes a graphical illustration showing examples of how changes in an estimate of Norm R_{expected} may affect the accuracy of CNV calling.

FIG. 5A includes a diagram illustrating an example configuration for training and/or implementing a machine learning model.

FIG. 5B includes a diagram illustrating an example configuration of a neural network.

FIG. 6 includes a graph that illustrates an example of the prediction accuracy of the different measures of a total signal intensity of a probe using the linear regression model.

FIG. 7 includes a graph that illustrates an example of the prediction accuracy of the total signal intensity of a probe using a random forest model trained for predicting the ENorm R as the response variable.

FIG. 8 includes a graph that illustrates an example of the prediction accuracy of different machine learning models.

FIG. 9 includes a graph that illustrates an example of the prediction accuracy of different machine learning models accepting different types of input for predicting the same response variable.

FIG. 10 includes a heatmap that shows a Pearson correlation of observed and predicted values of test data for different combinations of test data and sample-specific models predicting EuclidNorm.

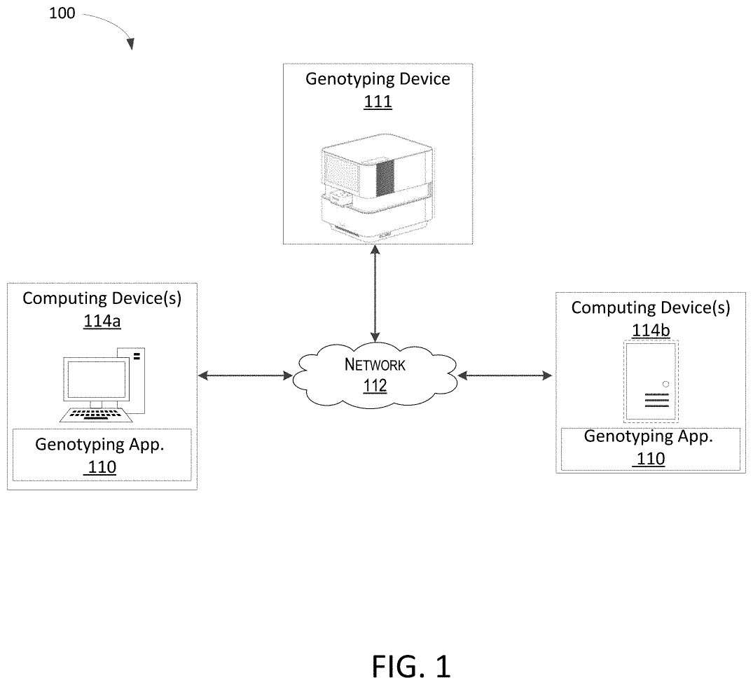

FIG. 1I includes another heatmap that shows a Pearson correlation of observed and predicted values of test data for different combinations of test data and sample-specific models predicting LRR.

FIG. 12 includes a graph that illustrates an example of an observed total signal intensity and a predicted total signal intensity of a probe signal.

FIG. 13 includes a graph that illustrates an example of a feature rank and a median feature influence on a random forest model for tested probe features.

FIG. 14 includes a heatmap that illustrates an example of spearman correlation of DNase mean rank with the predicted total probe signal intensity R from a random forest model trained using the normalized total intensity ENorm R.

FIG. 15 includes a graph that illustrates an example of the prediction accuracy of the different measures of the total signal intensity of the probe using the linear regression model that has been trained using a primer melting temperature (TM) as a single probe feature as input.

FIG. 16A includes a graphical illustration showing examples of signal separation for total signal intensity calculated using different embodiments described herein.

FIG. 16B includes a graphical illustration showing examples of signal separation for different genes or regions of probes of particular interest in a genome.

FIG. 16C includes two graphical illustrations showing a mean signal separation for each of the different copy numbers illustrated by the plots in FIG. 16A.

FIG. 16D includes two graphical illustrations showing bimodal distributions for each of the different copy numbers illustrated by the plots in FIG. 16A.

FIG. 17A is a flowchart depicting an example procedure for training a machine learning model to predict a total signal intensity of a probe signal.

FIG. 17B is a flowchart depicting an example procedure for predicting the total signal intensity of a probe signal and applying the predicted total signal intensity.

FIG. 18 is a block diagram of an example computing device.

### DETAILED DESCRIPTION

FIG. 1 illustrates a schematic diagram of a system environment (or “environment”) 100. The system environment 100 includes genotyping device 111, one or more computing device(s) 114a, 114b, and one or more network(s) 112. Computing devices 114a may include one or more client computing devices. Computing devices 114b may include one or more server devices or other remote computing devices.

The computing devices 114a, 114b and/or the genotyping device 111 may be capable of communication with one another via the network(s) 112. The network 112 may comprise any suitable network over which computing devices can communicate. The network 112 may include a wired and/or wireless communication network. Example wireless communication networks may be comprised of one or more types of RF communication signals using one or more wireless communication protocols, such as a cellular communication protocol, a WIFI communication protocol, and/or another wireless communication protocol. In addition, or in the alternative to communicating across the network. 112, the genotyping device 111 and/or the computing devices may bypass the network 112 and may communicate directly with one another.

The technology described herein may apply to a variety of genotyping devices 111, also referred to as genotyping scanners and genotyping platforms. The genotyping device 111 may include imaging systems like Illumina's BeadChip imaging systems such as the ISCAN™ system. The genotyping device 111 can detect fluorescence intensities of hundreds to millions of beads arranged in sections on mapped locations of image-generating chips. The image-generating chips of the genotyping device 111 may be equipped with internal probes designed to support quality control of the genotyping process. The probes may include capture probes, DNA probes, oligonucleotide probes, process probes, and/or other probes. A variety of process probes generate signals indicating the processing conditions and sample quality at different process steps of the genotyping process. Genotyping microarrays are also referred to as single-nucleotide polymorphism (SNP) arrays. The design of a genotyping array is based on the concept of hybridization technology.

The genotyping device 111 may include a processor that controls various aspects of the genotyping device 111, for example, laser control, precision mechanics control, detection of excitation signals, image capture, image registration, image extraction, and/or data output. The sample preparation can take two to three days and can include manual and/or automated handling of samples. The processor may generate image data comprising raw images or raw signals that have been excited on an image-generating chip and store the image data in memory. The genotyping device 111 may include a separate imaging circuit configured to generate the image data and provide the image data to the processor for being stored in memory.

The genotyping device 111 may capture raw images or raw signals on the mapped locations of the image-generating chips and transmit the raw images or raw signals in image data to one or more computing devices 114a, 114b, either directly or via the network 112. The computing devices 114a, 114b may receive the image data from the genotyping device 111 and perform further processing based on the image data.

The computing devices 114b may comprise a distributed collection of servers distributed across the network 112 and located in the same or different physical locations. Further, the computing devices 114b may comprise a content server, an application server, a communication server, a web-hosting server, or another type of server. The computing devices 114b may include one or more genotyping applications 110b that may be stored in computer-readable memory that, when executed by a processor, cause the computing devices 114b to perform as described herein. For example, the one or more genotyping applications 110b may cause the computing devices 114b to analyze the image data received from the genotyping device 111 to perform normalization of the signals received in the image data, clustering of the signals in the image data, and/or analyze genotype calling data, generated from the signals in the image data or otherwise received from the genotyping device 111, to perform genotype calls. For example, the computing devices 114b may receive raw data from the genotyping device 111 and may determine a nucleotide base sequence for a nucleic-acid segment and/or a variant thereof. The computing devices 114b may determine the sequences of nucleotide bases in DNA and/or RNA segments or oligonucleotides. The computing devices 114b may execute one or more applications capable of training and/or implementing one or more machine learning models to perform as described herein.

While the genotyping device ill is described separately from computing devices 114a, 114b, the genotyping device may be a computing device with imaging capabilities such that the genotyping device may perform processing on the image data, as described herein, directly on the genotyping device itself. The genotyping device 111 may include one or more genotyping applications 110c that may be stored in computer-readable memory that, when executed by a processor, cause the genotyping device 111 to perform as described herein. For example, the one or more genotyping applications 110c may cause the genotyping device 111 to analyze the image data generated thereon to perform normalization of the signals received in the image data, clustering of the signals in the image data, and/or analyze genotype calling data, generated from the signals in the image data, to perform genotype calls. For example, the genotyping device 111 may generate raw image data and may determine a nucleotide base sequence for a nucleic-acid segment and/or a variant thereof. The genotyping device 111, using the one or more genotyping applications 110c thereon, may determine the sequences of nucleotide bases in DNA and/or RNA segments or oligonucleotides. The genotyping device 111 may execute one or more genotyping applications 110c capable of training and/or implementing one or more machine learning models to perform as described herein. The genotyping applications 110c may be the same as, or different from, the genotyping applications 110b residing on the computing devices 114b. One or more portions of the genotyping applications may be distributed across the genotyping device 111, the computing devices 114b, and/or one or more other computing devices.

The computing devices 114a may generate, store, receive, and/or send digital data. For example, the computing devices 114a may receive image data from the genotyping device 111 and/or computing devices 114b. The computing devices 114a may communicate with the computing devices 114b to receive variant call file comprising nucleotide base calls and/or other metrics, such as a call-quality, a genotype indication, and/or a genotype quality. The computing devices 114a may receive input from a user and/or communicate with the computing devices 114b to provide instructions in response to the input. The computing devices 114a may present or display image data or other information pertaining to genotype calling within a graphical user interface to a user associated with the computing device 114a. The computing devices 114a may provide instructions to the computing devices 114b to enable the computing devices 114b to train and/or implement one or more machine learning models, as described herein.

The computing devices 114a illustrated in FIG. 1 may comprise various types of client devices. In examples, the computing devices 114a may include non-mobile devices, such as desktop computers or servers, or other types of client devices. In other examples, the computing devices 114a may include mobile devices, such as laptops, tablets, mobile telephones, or smartphones.

As further illustrated in FIG. 1, the computing devices 114a may include one or more genotyping applications 110a that may be stored in computer-readable memory that, when executed by a processor, cause the computing devices 114a to operate as described herein. The genotyping application 110a may include a web application or a native application stored and executed on the computing devices 114a (e.g., a mobile application, desktop application). The genotyping application 110a may include instructions that (when executed) cause one or more computing devices 114a to receive data from the genotyping device 111 and/or computing devices 114b and present, for display at the computing device 114a, data for display to the user. Furthermore, the genotyping application 110 may instruct the computing device 114a to display a visualization of graphs, clusters, metrics, and/or contribution measures for genotype calling. The genotyping application 110a may perform image processing and/or implement, train, and/or run machine learning models, as described herein. The genotyping applications 110a may be the same as, or different from, the genotyping applications 110b, 110c residing on the computing devices 114b and/or genotyping device 111. One or more portions of the genotyping applications may be distributed across the computing devices 114b, the genotyping device 111, the computing devices 114b, and/or one or more other computing devices.

The methods, systems, and apparatus described herein may be used for analyzing any of a variety of objects. An example object comprises solid supports or solid-phase surfaces with attached analytes. The methods, systems, and apparatus described herein may be used with objects having a repeating pattern of analytes in an x-y plane. An example is a microarray having an attached collection of cells, viruses, nucleic acids, proteins, antibodies, carbohydrates, small molecules (such as drug candidates), biologically active molecules or other analytes of interest.

An increasing number of applications have been developed for arrays with analytes having biological molecules such as nucleic acids and polypeptides. Such microarrays may include deoxyribonucleic acid (DNA) or ribonucleic acid (RNA) probes, which are specific for nucleotide sequences present in humans and other organisms. In certain applications, for example, individual DNA or RNA probes may be attached at individual analytes of an array. A test sample, such as from a known person or organism, can be exposed to the array, such that target nucleic acids (e.g., gene fragments, mRNA, or amplicons thereof) hybridize to complementary probes at respective analytes in the array. The probes can be labeled in a target specific process (e.g., due to labels present on the target nucleic acids or due to enzymatic labeling of the probes or targets that are present in hybridized form at the analytes). The array can then be examined by scanning specific frequencies of light over the analytes to identify which target nucleic acids are present in the sample. In an example, the genotyping device 111 of FIG. 1 may receive the microarray and perform the scanning of the frequencies of light over the analytes to generate image data comprising raw images or signals that may be processed to identify target nucleic acids.

Genotyping application 110a herein maybe implemented to screen for the presence of a genetic locus of interest in a target nucleic acid sample. A locus of interest in a typical genotyping protocol, and as disclosed herein, may include, without limitation, polymorphs (e.g., single nucleotide polymorphs (SNPs), indels), short tandem repeats (STR), copy number variants (CNV), germline variants, methylation sites (e.g., CpG islands), and exogenous sequences (e.g., virus). Target nucleic acid samples herein may include polynucleotides of any length, and may be derived from any number of genetic sources including from human or non-human organisms, and from individual organisms or organism populations. Samples herein may be obtained from wide variety of genetic materials—e.g., gDNA, mtDNA, mRNA, cDNA transcribed from mRNA, non-coding RNA, and small RNA, polynucleotide conjugates, analogues, and amplicons.

Any of a variety of arrays (also referred to as “microarrays”) known in the art can be used in a method or system set forth herein, including, e.g., assay work flows for SNP genotyping. Image-generating chip arrays provide a convenient format for assaying SNPs, particularly at commercial scale. An example workflow may begin with accession and extraction of a DNA sample, either from single cell source or a tissue sample. The extracted DNA sample may be amplified, usually off-chip in solution, and the amplicon output is then subjected to controlled enzymatic fragmentation. The processed DNA sample is loaded onto the image-generating chip and subjected to hybridization using locus specific oligo probes functionalized on the chip substrate. Allelic specificity of hybridized DNA is conferred by enzymatic base extension at the 3′ end of the probe. Base extensions are applied fluorescent labels, imaged under excitation, and allele signal intensity data is used to perform genotype calling. An array may be functionalized with an individual probe or a population of probes. In the latter case, the population of probes at each analyte is typically homogenous having a single species of probe. For example, in the case of a nucleic acid array, each locus specific probe may be amplified to yield multiple nucleic acid molecules each having a common sequence. However, in some implementations the population of probes at a given reaction site of an array can be heterogeneous. Similarly, protein arrays can be functionalized with a single protein probe or a population of protein probes typically, but not always, having the same amino acid sequence. The probes can be attached to the surface of an array for example, via covalent linkage of the probes to the surface or via non-covalent interaction(s) of the probes with the surface.

Example arrays include, without limitation, a BeadChip Array available from ILLUMINA, INC. (San Diego, Calif.) or others such as those where probes are attached to beads that are present on a surface (e.g. beads in wells on a surface). Further examples of commercially available microarrays that can be used include, for example, an AFFYMETRIX GENECHIP microarray or other microarray synthesized in accordance with techniques sometimes referred to as VLSIPS™ (Very Large Scale Immobilize Polymer Synthesis) technologies. A spotted microarray can also be used in a method or system according to some implementations of the present disclosure. An example spotted microarray is a CODELINK Array available from AMERSHAM BIOSCIENCES. Another microarray that is useful is one that is manufactured using inkjet printing methods such as SUREPRINT Technology available from AGILENT TECHNOLOGIES.

During a genotyping operation, optical signals provided by the sample are observed through an optical system. Various types of imaging may be used with embodiments described herein. For example, embodiments may be configured to perform at least one of fluorescent imaging, epi-fluorescent imaging, and total-internal-reflectance-fluorescence (TIRF) imaging. In particular embodiments, the sample imager is a scanning time-delay integration (TD) system. Furthermore, the imaging sessions may include “line scanning” one or more samples such that a linear focal region of light is scanned across the sample(s). Imaging sessions may also include moving a point focal region of light in a raster pattern across the sample(s). Alternatively, one or more regions of the sample(s) may be illuminated at one time in a “step and shoot” manner.

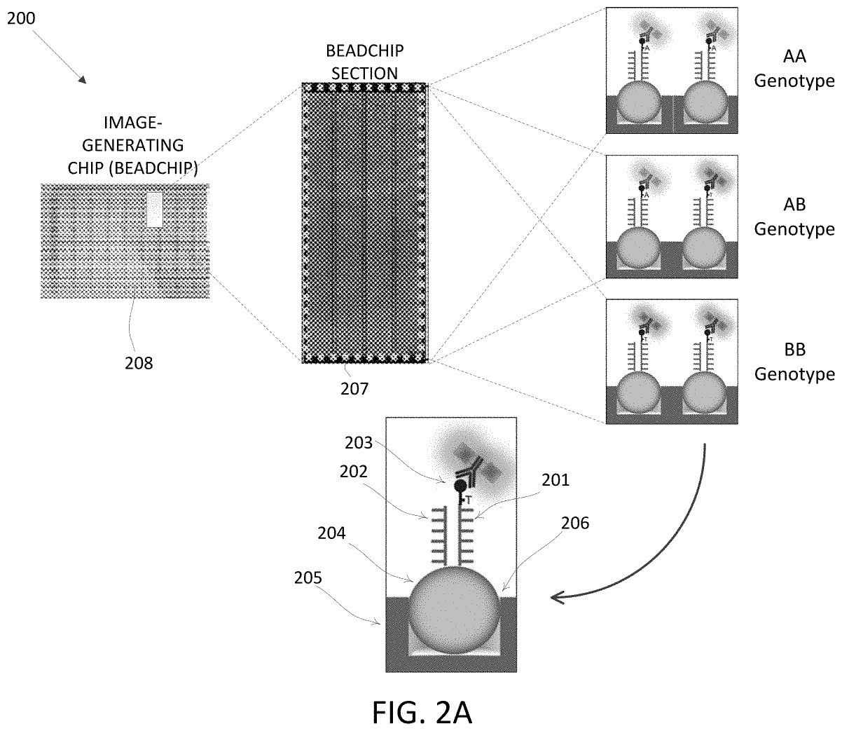

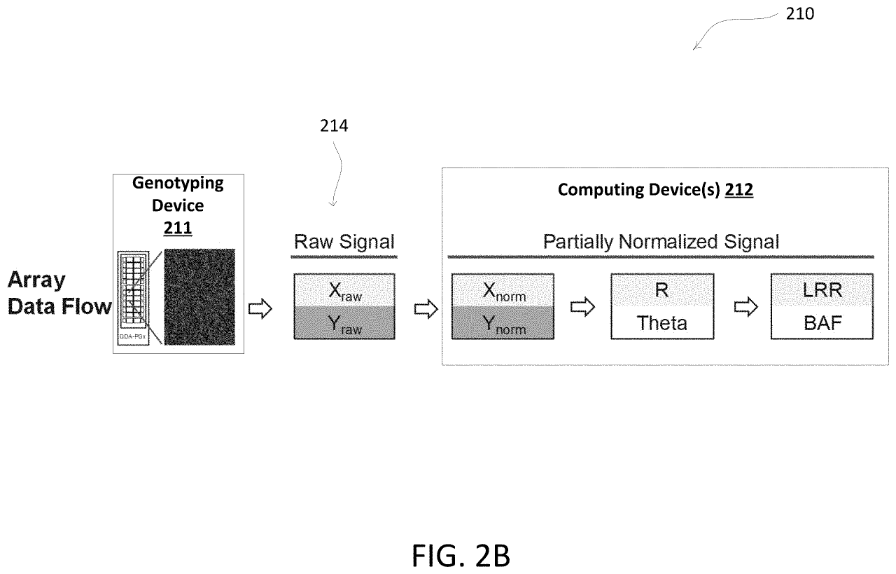

FIG. 2A is an illustration 200 indicating capture probes 201 that may be positioned on an image-generating chip 208 in a genotyping device, such as the genotyping device 111 shown in FIG. 1. The image-generating chip 208 may also referred to as BeadChips or BeadArrays. The image-generating chip 208 may include multiple sections 207 that are arranged in columns and rows on the image-generating chip 208. For example, the image-generating chip 208 may have 12, 24, 96 or more sections, each of which may have a separate DNA sample. Beads 204 (e.g., glass beads) may be positioned in wells 206 (or holes) at known locations on the surface of the image generating chip 208. Numerous (e.g., thousands or more) different types of oligonucleotide probes 201 may be attached to the beads 204 at random known locations and replicated many times (up to 10 times or more) on the image-generating chip 208. In some implementations the Illumina Infinium™ platform may be used, which includes hundreds of thousands to millions of micro-wells on a BeadChip, and microbeads are distributed in the microwells. The microbeads have diameters of roughly 3 μm. DNA samples are processed, amplified, and provided to the BeadChip. Each bead 204 is covered with hundreds of thousands of copies of a specific oligonucleotide that acts as capture sequences targeting different SNPs. Allelic specificity of hybridized DNA 202 is conferred by enzymatic base extension at the 3′ end of the probe. Base extensions are applied fluorescent labels 203, imaged under excitation, and allele signal intensity data is used to perform genotype calling.

The illustration 200 shows three different example samples, each with a corresponding pair of probes 201 for hybridizing respective wild-type and mutant alleles at a biallelic loci. Each probe is grafted to a respective microbead on the surface of a section 207 of the image-generating chip 208. The capture probes 201 may be antisense oligonucleotide probes as they may be designed to target specific positions in the complementary DNA sense sequences 202 of a sample (also referred to as DNA templates). The capture probes 201 may be of different lengths. In one example, the capture probes 201 may comprise a 50 bp probe. The capture probes 201 may be extended with single fluorescently labeled bases 203. In the example of FIG. 2A a thymine ligated with a red channel fluorophore may be used with a wild-type (A) antisense oligo probe, and an adenine ligated with a green channel fluorophore may be used with a mutant (B) antisense oligo. The fluorescently labeled bases 203 may comprise fluorescently labelled dideoxynucleotides triphosphates (ddNTPs). After a sample allele hybridizes to a respective antisense oligo probe, the probe may be extended with a fluorescently labelled ddNTP that emits a colored signal under excitation (e.g., either a red or green signal) depending on the allele.

FIG. 2B illustrates a system 210 that may be implemented for imaging the capture probes 201 and processing the image signals for genotype calling based on signals detected in a particular channel from the fluorescent labels. As shown in FIG. 2B, the system 210 may include a genotyping device 211. As described herein, the genotyping device 211 may be a device capable of generating image data, such as the ILLUM INA ISCAN system, for example. The nucleotide corresponding to the allele may be measured based on the signals in the image data. The genotyping device 211 device may generate different image data for different sections of the image-generating chip.

To detect the two alleles the one wild-type (A) and the other mutant type coding a known SNP of interest, two of the probes 201 (oligonucleotides) are synthesized to capture each of the two alleles for the SNP. The fluorescent signal intensity of each probe that is detected in the image data represents the signal strength for each allele. When the nucleotides corresponding to a specific SNP are measured by the genotyping device 211, the intensity and the colors of the signals in the image will indicate the quantity and identity of the two alleles in the genetic sample.

In some implementations the ILLUMINA INFINIUM or GOLDEN GATE microarrays may be used to provide the genotyping data. These and other platforms produce two-colored readouts (e.g., one color for each allele) for each single nucleotide polymorphism in the genotyping study. Intensity values for each of two color channels may convey information about the allele ratio at a locus. Each color channel may correspond to a different allele, such as an allele A and an allele B, for example.

Many applications incorporate values for a large number of samples (hundreds to tens of thousands) to ensure significant statistical representation. When these values are appropriately normalized and plotted, distinct patterns or clusters emerge, in which samples that have identical genotypes at an allele locus exhibit similar signal profiles (A and B values). In contrast, samples with differing genotypes will appear in separate distinct clusters. For diploid organisms, biallelic loci are expected to exhibit three clusters: AA, AB, and BB. In the example of FIG. 2A, an AA readout indicates that the source organism for the genetic sample is of a normal genotype, having only wild-type (or normal) alleles at the subject loci; an AB readout indicates the source organism is of a heterozygous genotype, having one wild-type and one mutant allele; and a BB readout indicates the source organism is of a homozygous genotype, having only mutant alleles.

Referring to FIG. 2B, the raw x and y signals 214 may represent raw probe intensity values of different colored signals (e.g., red and green signals) of sections of the image-generating chip. The raw x signal X_{raw} may indicate the raw probe intensity value measured by the genotyping device 111 for the allele A. The raw y signal Y_{raw} may include the raw probe intensity value measured by the genotyping device 111 for the allele B. The raw x and y signals 214 may be sent to a computing device 212 for being analyzed to detect the alleles. In one example, the computing device 212 may be a separate device capable of processing the raw x and y signals 214. In another example, the genotyping device 211 may be an imaging device or system that may include or be included in the computing device 212, such that the raw x and y signals 214 may be processed on-device, as described herein, without being communicated to a separate device.

The raw x and y signals 214 may include varying intensities detected for the probes (e.g., capture probes, DNA probes, oligonucleotide probes, etc.), which may be reported at high intensities, low intensities, and/or background level intensities. Signal intensity emitted from a probe is subject to variations in DNA sample preparation methods, sources of a sample, or tissue type. Signal intensities can also vary because of variability in which individuals perform the assay. Variations in genotyping devices or scanners can also impact signal intensities emitted by probes. Because of these variations, the image data comprising the raw x and y signals 214 that are generated from these probes may not be assessed based on the absolute values.

The raw x and y signals 214 may be sent to the computing device 212 for pre-processing of the signal intensities. The computing device 211 may receive the raw x and y signals 214 and normalize the intensity values prior to performing additional processing, such as clustering and/or genotype calling. The normalization procedure may be performed according to one or more normalization procedures, such as those implemented by ILLUMINA, INC.'s BEADSTUDIO software, for example. The computing device 211 may normalize X_{raw} and Y_{raw} to obtain the normalized values X_{normalized} and Y_{normalized}. A total intensity value for the raw or normalized signal may be indicated by a value R, which may be calculated as defined in

Equation 1:

R=X+Y Equation 1:

where R may be a raw or normalized probe intensity value that is calculated as the total intensity value of a signal based on a raw or normalized X intensity value and a raw or normalized Y intensity value.

The computing device 212 may apply a cluster algorithm to the fluorescent levels to form a cluster that distinguishes samples for better visualization and/or perform genotyping. The computing device 211 may polar transform X_{normalized} and Y_{normalized} into R and Theta coordinates for clustering, as further described herein. The computing device 211 may also, or alternatively, derive a Log R ratio (LRR) and B-allele frequencies (BAF) values from R and Theta, as further described herein, to perform CNV calling or other genotype calling.

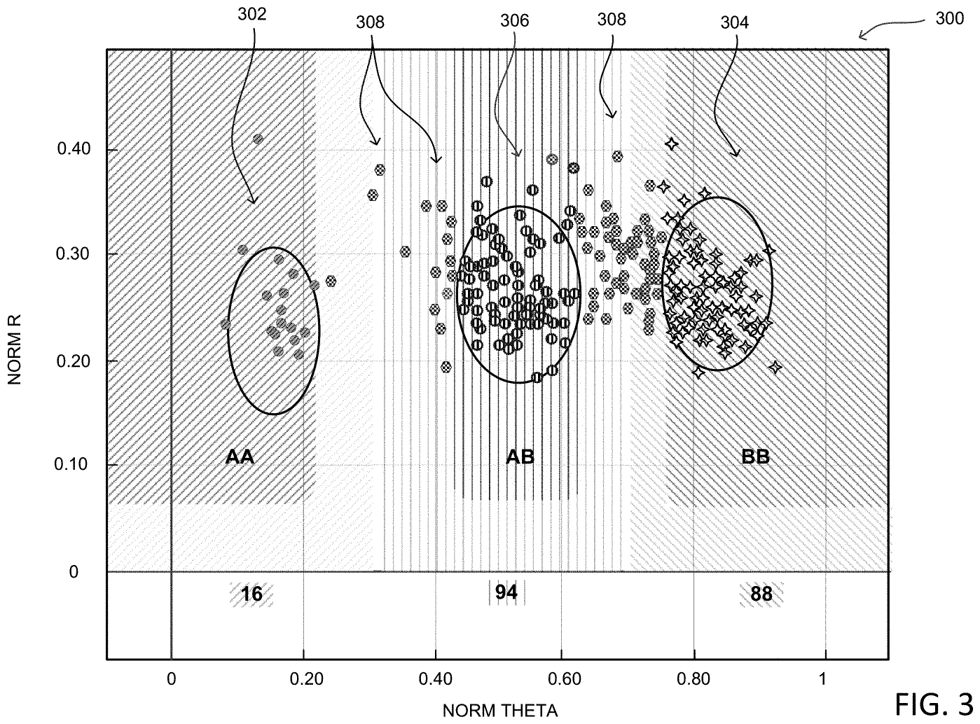

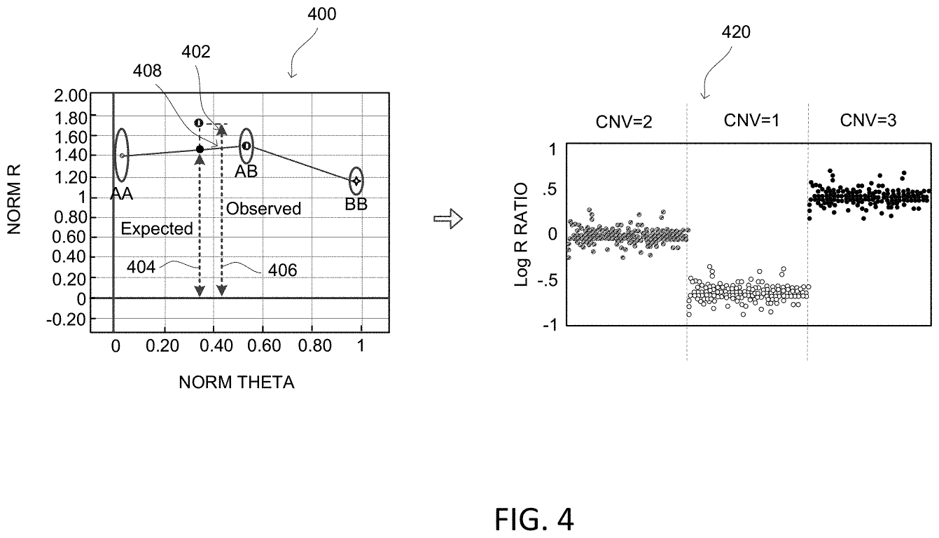

FIGS. 3 and 4 illustrate how clustering and genotype calling, respectively, may depend on effective normalization of the raw x and y signals 214 generated by the genotyping device 211. FIG. 3 is a graph 300 illustrating clusters of data based on data from a two-channel microarray platform transformed to polar coordinates. The x and y signals may represent an intensity value for allele A and allele B channels, respectively, that are obtained from a collection of DNA samples that may serve as input values for developing clusters. The level of fluorescent intensity of each probe represents the signal strength for each genotype. After measuring fluorescent levels of the two probes from multiple samples, a cluster algorithm is applied to the fluorescent levels to form a cluster that distinguishes samples into AA, AB and BB clusters representing corresponding genotypes of the SNP.

The A and B channels in the graph 300 illustrate clusters of signals representing A and B genotypes that are based on normalized signals (e.g., X_{normalized} and Y_{normalized}), though a similar graph may be generated using raw signals (e.g., X_{raw} and Y_{raw}). Clusters corresponding to these signals can be characterized by five parameters: mean of A intensities, mean of B intensities, standard deviation of the A intensities, standard deviation of B intensities, and covariance of A and B intensities. In many samples, the covariance parameter is significant for the AB cluster, because the AA and BB clusters mostly lie along their respective axis. The clustering may be performed by a clustering algorithm, such as ILLUMINA, INC.'s GENTRAIN 3.0 clustering algorithm, for example. When the data of different genotypes are shown in a two-color space, they form distinguishable clusters.

To simplify the clustering process or the visualization thereof in the graph 300, the A and B intensities have been transformed into two values, labeled normalized R and normalized Theta. The y-axis of the graph 300 includes normalized R, which is computed as defined in Equation 1 herein. The x-axis of the graph 300 includes normalized Theta that quantifies the relative amount of signal measured by the A and B intensities. Normalized Theta is computed as defined by Equation 2:

Norm Theta=2π⁻¹ arctan(AB⁻¹) Equation 2:

where, again, A represents the normalized probe intensity value for allele A (e.g., X_{normalized}), and B represents the normalized probe intensity value for allele B (e.g., Y_{normalized}). Although the graph 300 includes normalized signals represented by normalized R and normalized Theta, a similar graph may also, or alternatively, be generated for R and Theta based on raw x and y signals (e.g., X_{raw} and Y_{raw}) using Equation 1 and Equation 2.

The clusters 302 correspond to genotype AA and may be designated with first points on the graph 300. The Theta value for the clusters 302 are between about 0 and about 0.21. The clusters 304 correspond to genotype BB and may be designated with second points on the graph 300. The Theta value for the clusters 304 are between about 0.78 and about 1. The clusters 306 correspond to genotype AB and may be designated with third points on the graph 300. The Theta value for the clusters 306 are between about 0.42 and about 0.62. The samples 308 in between clusters may not be assigned a genotype.

In the plot of signals in graph 300, the genotype for samples 308 may be unable to be determined. For example, a cause of the genotype for samples 308 being unable to be determined may be the total DNA in a sample, which may affect the total intensity of the probe signal. For example, in saliva, a high proportion of DNA content may be microbial. This confounder breaks down certain normalization procedure and may results in ambiguous genotype calls.

In addition to affecting the clustering of signals, effective normalization of the raw x and y signals generated by genotyping devices may affect genotype calling. One example of ambiguous genotype calls may be observed in call number variant (CNV) calling. Accurate CNV calling may depend on a reference dataset of total probe signal. For CNV calling, Norm R and Norm Theta may be compared to the reference dataset by computing a Log R ratio (LRR). LRR is the normalized measure of signal intensity for each SNP marker in an array. LRR is calculated taking the log2 of the ratio between the observed signal and expected signal for two copies of the genome, and can be expressed in Equation 3:

$$Log R Ratio ( L R R ) = {log}_{2} ( \frac{{NormR}_{observed}}{{NormR}_{expected}} ) \qquad Equation 3$$

where Norm R_{observed} is the normalized R value representing the intensity of the observed sample in the image data, and Norm R_{expected} is a predefined value of the normalized intensity level of the signal that is expected based on a reference dataset. Norm R_{expected} is an average value of the normalized intensity level of the signal generated across multiple samples to estimate the expected value. This average value for Norm R_{expected} may be calculated based on semi-manually determined (e.g., with some user interaction) clusters of samples in reference datasets that are independent of the samples being used to calculate Norm R_{observed}. This calculation of Norm R_{expected} may be separately calculated based on reference datasets generated at different genotyping devices, so as to generate the expected value at the specific device. As the clustering and calculation is performed semi-manually and independently for each device, Norm R_{expected} may be biased by the individual and/or the type of genotyping device.

The LRR value may be used to call CNVs. Thus, accurate CNV calling may depend on the estimate of an expected total signal intensity R of the probe signal (e.g., which is determined from a reference dataset), such as Norm R_{expected}, for example. Changes in this value can lead to false positives and false negatives. FIG. 4 includes a graphical illustration of how changes in an estimate of Norm R_{expected} may affect the accuracy of CNV calling. As shown in FIG. 4, graph 400 illustrates a difference 408 between an observed Norm R intensity value Norm R_{observed} 406 in a sample 402 and an expected Norm R intensity value Norm R_{expected} 404. Graph 420 shown in FIG. 4 illustrates how CNV calling is based on the LRR. As shown in the graph 420, an LRR value of within a range of 0 is called with a first CNV value (e.g., CNV=2), an LRR value of within a range of 0.5 is called with a second CNV value (e.g., CNV=3), and an LRR value of within a range of −0.5 is called with a third CNV value (e.g., CNV=1). As can be shown from the graphs 400, 420 in FIG. 4, the difference 408 between the observed Norm R intensity value Norm R_{observed} 406 in the sample 402 and the expected Norm R intensity value Norm R_{expected} 404 can affect the ultimate CNV calling for the sample 402. As such, changes in the expected Norm R intensity value Norm R_{expected}, may change the ultimate CNV call.

Though the total signal intensity R of a probe signal may be normalized in an attempt to improve the use or application of the total signal intensity R in genotyping applications, the normalization may rely on external reference datasets to compute an expected Norm R intensity value (Norm R_{expected}), which may be less reliable for normalizing signals for some samples than for others. External controls may include samples which are known to produce a predetermined result when analyzed and are often included as points of reference that does not fall within the experimental data set. As reference points, the external controls can be used to determine one or more parameters of a selected function which is used to normalize an unknown data set. Disadvantages to using these external controls for performing such normalization may include difficulties in keeping external controls constant over time and/or across samples.

Studies have identified that the expected signal intensity from a probe varies across samples. For example, the expected signal intensity of a sample may vary across individuals. One example of expected signal intensity of a sample varying across individuals is shown in the following article by Diskin, Sharon J., el al., entitled “Adjustment of genomic waves in signal intensities from whole-genome SNP genotyping platforms”, Nucleic acids research 36, no. 19 (2008): e126-e126. Genomic waves have also been observed in LRR data. This variation is independent of copy number variation, and the amplitude and phase of the waves are sample-specific. These waves may be associated with guanine-cytosine (GC) content of the samples. Thus, correcting the expected signal intensity for GC content may improve the specificity of CNV calling.

Embodiments are described herein for utilizing machine learning models to generate a predicted total signal intensity R_{predicted} of a probe signal based on sample-specific image data. The predicted total signal intensity R w of a probe signal may be a total raw signal intensity or a normalized signal intensity. As the predicted total signal intensity R_{predicted} of the probe signal is based on sample-specific image data, the predicted total signal intensity R_{predicted} of the probe signal may be more accurate than an expected Norm R intensity value Norm R_{predicted} that is based on external data. The predicted total signal intensity R_{predicted} may be used in downstream genotyping applications to improve the accuracy of the application. Additionally, the use of a trained machine learning model to generate the predicted signal intensity R_{predicted} may allow for more effective on-device processing at the genotyping device. For example, the on-device processing may generate the predicted signal intensity R_{predicted} without the use of a reference dataset (e.g., to calculate Norm R_{expected} for use in calculating LRR). If the reference dataset is implemented (e.g., to calculate Norm R_{expected}) in generating the normalized total signal intensity R of a probe signal, the reference dataset may be received from an external device or the image data may be sent to the external device at which the reference dataset is stored and additional processing may be implemented to calculate Norm R_{expected} from the reference dataset.

The raw or normalized probe intensity that is based on the sample-specific image data of the probe signal may be used to train the machine learning model to generate different response variables. The raw or normalized probe intensity may be computed as non-standard measures of total intensity (e.g., Raw R, ENorm R, etc.), which may be used to train the models described herein. Norm R and LRR may be examples of measures that may be used for genotyping and/or CNV calling. The response variables may be different predicted total signal intensity R_{predicted} values. TABLE 1 below provides raw and normalized total signal intensities R for the probe signal. The probe intensities in TABLE 1 may represent different types of response variables output as the predicted total signal intensity R_{predicted} values by the machine learning models. A definition for each of the raw and normalized total signal intensities R are provided and may be based on the sample-specific image data of the probe signal comprising the raw x and y signals that are received from the genotyping device.

TABLE 1

| Probe Intensity Measures | Definition |
|---|---|
| Norm R | xₙₒᵣₘ + yₙₒᵣₘ |
| Raw R | x_{raw} + y_{raw} |
| ENorm R | \sqrt{xₙₒᵣₘ² + yₙₒᵣₘ²} |
| LRR | log₂ (R_{observed}/R_{expected}) |

As shown in TABLE 1, a raw probe intensity Raw R may be generated based on the raw x and y signals (e.g., X_{raw} and Y_{raw}) received in the sample-specific image data for the probe and may be used to train the machine learning models to predicted raw signal intensity Raw R_{predicted} of a probe signal for Raw R. Additionally, or alternatively, a normalized probe intensity (e.g., Norm R, ENorm R, or LRR) may be generated based on the normalized probe intensity value for the signals received in the sample-specific image data for the probe (e.g., X_{normalized} and Y_{normalized}) and the normalized probe intensity may be used to train the machine teaming models to predicted total signal intensity R_{predicted} of a probe signal for the normalized value (e.g., Norm R_{predicted}, ENorm R_{predicted}, or LRR_{predicted}).

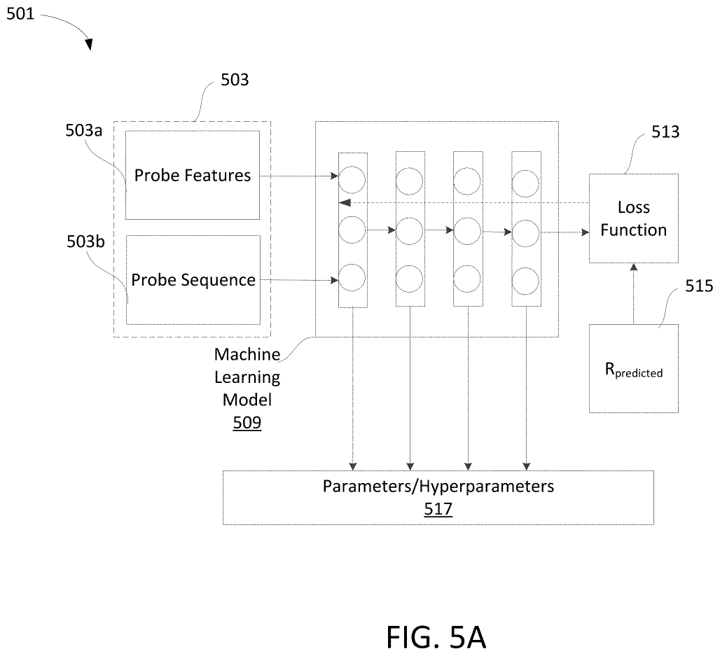

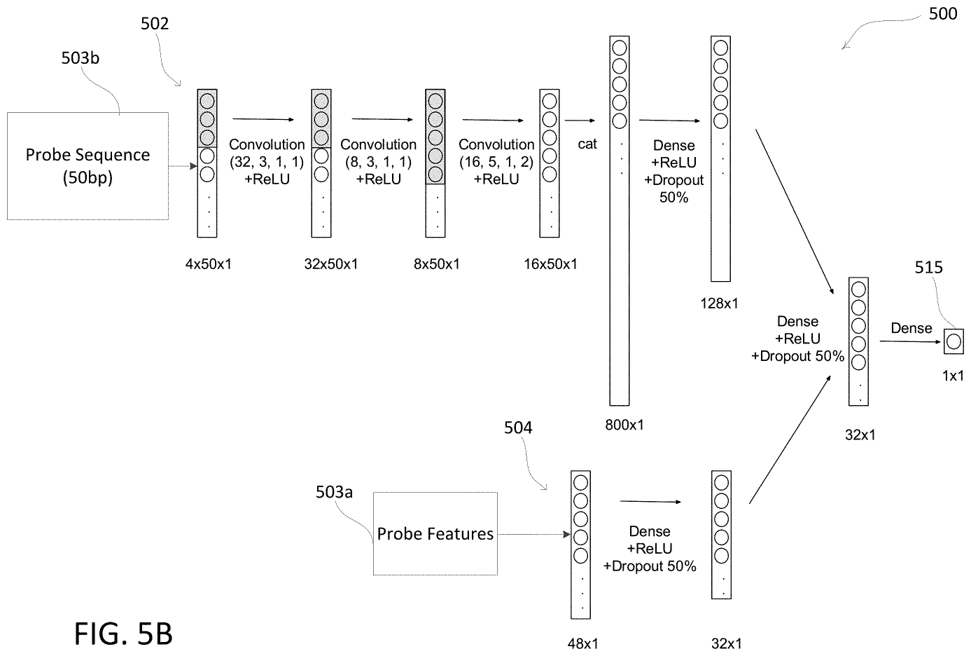

FIG. 5A is a schematic illustration of an example system environment 501 for training a machine learning model 509 to predict the observed signal intensity R_{observed} and generate a predicted total signal intensity R_{predicted} 515 of a probe signal. The machine learning model 509 may receive an input 503 that includes training data for training the machine learning model 509. The input 503 may include one or more types of input data 503a, 503b. The input 503a may include probe features. As the fluorescently labeled target sequences created from source samples generates a signal that depends on the hybridization conditions, the machine learning model 509 may be trained with one or more probe features 503a being received as an input 503 to account for one or more of these conditions in determining the predicted total signal intensity R_{predicted} of a probe signal. Thus, the machine learning model 509 may be trained to generate the predicted total signal intensity R_{predicted} of a probe signal based on the combination of the probe features by the machine learning model 509. The machine learning model 509 may be trained to update parameters and/or tune hyperparameters 517 that may be used to determine a predicted probe intensity value R_{predicted} 515 based on the combination of the probe features. Hyperparameters may be variables that govern the training process itself. These variables may not be directly related to the training data but may be configuration variables. The parameters may change during training, while hyperparameters may remain constant during a training session using the training data.

As further described herein, the machine learning model 509 may also, or alternatively, receive one or more additional inputs. For example, the input 503b may include a probe sequence, or a portion thereof. The probe sequence may be a type of probe feature, but may be received separately by the machine learning model 509. The probe sequence in the input data may be received as a vector, tensor, textual data, or another sequence of data. The sample-specific image data may be associated with a sample relating to a single individual. The sample-specific image data may be separated into training data, test data, and/or validation data. The sample-specific image data may include image data based on apriori known probe sequences or sequence derived features that may be sued to model the signal. The sample-specific image data may be image data related to raw and/or normalized values in a format (e.g., vector, tensor, or other format) capable of being received by the machine learning model 509. In one example, the sample-specific image data may be pre-processed to generate a one-hot encoded probe sequence for a number of base pairs that indicates different values in the probe sequence. The machine learning model 509 may also, or alternatively, receive the probe sequence input 503b as image data or in another format capable of being received at the machine learning model 509. Though multiple forms of input 503 are provided as examples, the machine learning model 509 may be trained on and/or implemented using one or more types of input, as described herein.

The machine learning model 509 may be trained, using the probe sequence 503b received as input and sample specific R_observed as target, to train parameters 517 that may be used to determine a predicted probe intensity value R_{predicted} 515. During the training process, the parameters 517 of the machine learning model 509 may be updated. The parameters 517 may include weights, biases, or coefficients of one or more layers, nodes, or functions of the machine learning model 509. The predicted probe intensity value R_{predicted} 515 may be a predicted total signal intensity of the signal associated with the sample for the probe. The predicted probe intensity value R_{predicted} 515 may represent a raw probe intensity value or a normalized probe intensity value. The normalized probe intensity value may be generated during a preprocessing step. The normalized probe intensity value may be calculated as the sum of the normalized x and y intensities, the Euclidean norm of the normalized x and y intensities, or a Log R ratio. The parameters 517 may be updated during the training process to adjust the weights allocated to the one or more probe features 503a for a given sample. This may result in the machine learning model 509 being trained to identify the relative influence of different probe features 503a, or categories thereof, on the probe intensity for a particular sample. The parameters 517 may also, or alternatively, be updated during the training process to adjust the weights allocated to the probe sequence 503b. This may result in the machine learning model 509 being trained to identify information contained in the probe sequence.

Different types of machine learning models 509 may be trained and/or implemented for generating the predicted total signal intensity R_{predicted} 515 of a probe signal. For example, the machine learning model 509 may include a linear regression model, a random forest model, a neural network, or another form of machine learning model. Each machine learning model 509 may include a machine learning algorithm that may be implemented on one or more computing devices. The machine learning model 509 may include a combination of different types of machine learning models, such as a combination of different types of neural networks. The machine learning model 509 may receive the probe features 503a as input data and output the predicted total signal intensity R_{predicted} of a probe signal as the response variable based on the probe features 503a.

When a linear regression model is implemented as the machine learning model 509, the linear regression model may assume a linear relationship between the probe features 503a that are received as input data and generate the predicted total signal intensity R_{predicted} of a probe signal as output. The linear regression model may assign a coefficient as a scale factor to each input value 503. The parameters 517 of the linear regression model may include the slope of the linear regression model. One additional coefficient may be added that may be referred to as the intercept or the bias coefficient, which may also be a parameter 517 that may be trained. The linear regression model may include a simple linear regression model or an ordinary least squares linear regression model. The linear regression may use backpropagation-based gradient updates and/or gradient descent techniques, such as batch gradient descent, Stochastic Gradient Descent (SGD) (e.g., synchronous SGD or asynchronous SGD), and/or mini-batch gradient descent. The linear regression model may be trained by calculating the loss from the output of the linear regression model to a target predicted total signal intensity R_{predicted} 515 via a loss function 513. The loss function 513 may be implemented to update the parameters 517 using backpropagation-based gradient updates and/or gradient descent techniques. Other examples of regression models that can be applied include K-nearest neighbors (KNN), Gaussian process.

When a random forest model is implemented as the machine learning model 509, the random forest model may include multiple decision trees, with each individual decision tree in the random forest acting as a predictor. Each decision tree will generate an output and the output is considered on a majority voting or averaging for regression, respectively. The number of trees used and the maximum depth of the trees may be tuned to reduce overfitting. When the random forest model is implemented, the random forest model may receive the probe features 503a as input data 503 and generate the predicted total signal intensity R_{predicted} 515 of a probe signal as output based on the aggregation of the probe features by the model. During training, the output of the random forest is compared with ground truth intensities and a prediction error may be calculated based on the loss function 513 to update the parameters 517. The parameters 517 of the trained random forest may be stored for use in predicting a total signal intensity R_{predicted} 515. The parameters 517 of the random forest model may include a number of decision trees, a maximum number of feature used, a maximum depth of a tree, a minimum impurity decrease per node split, and/or a minimum number of samples required to be at a leaf node.

When the machine learning model 509 implements one or more neural networks, each neural network may comprise one or more types of neural networks for receiving one or more inputs 503 to generate the predicted total signal intensity R_{predicted} 515 of a probe signal as output. For example, the neural network may include one or more layers of nodes or functions that may be trained, as described herein. For example, the layers may include one or more input layers, one or more hidden layers, and/or one or more output layers. The neural network may include a fully-connected neural network comprising fully-connected dense layers, a convolutional neural network (CNN) comprising convolutional layers, and/or a combination of convolutional layers and dense layers. When the neural network is implemented, an input layer of the neural network may receive the probe features 503a as input data 503 and the output layer may generate the predicted total signal intensity R_{predicted} 515 of a probe signal as output. The parameters 517 may include weights and biases of the machine learning model 509. The hyperparameters 517 may include a number of epochs, a batch size, a window size, a number of layers, and/or a number of nodes in each layer, for example. The parameters 517 of the neural network may be tuned during the training process to generate the predicted total signal intensity R_{predicted} 515 of a probe signal for a normalized value (e.g., Norm R_{predicted}, ENorm R_{predicted}, or LRR_{predicted}). The neural network may be trained using backpropagation-based gradient updates and/or gradient descent techniques, such as batch gradient descent, SGD (e.g., synchronous SGD or asynchronous SGD), and/or mini-batch gradient descent. During training, a prediction error may be calculated based on the loss function 513 to update the parameters 517. The parameters 517 of the trained neural network may be stored for use in predicting a total signal intensity R_{predicted} 515.

The training of the machine learning model 509 may be performed one or more times. The training may be performed by initializing one or more parameters 517 of the machine learning model 509, accessing the training data, inputting the training data into the machine learning model 509, and/or training the machine learning model 509 using the loss function 513 to achieve a target output 515. An optimizer may be implemented along with the loss function 513 to update the parameters and/or hyperparameters 517. During training, the parameters 517 may be updated (e.g., via gradient descent and associated back propagation) and the training process may be iterated until an end condition is achieved. The end condition may be achieved when the output of the machine learning model 509 is within a predefined threshold of the target output.

After the training process is complete, the trained parameters and/or hyperparameters 517 may be implemented by a machine learning model in an operating or production process. During the operating or production process, the trained machine learning model may receive input data and use the trained parameters and/or hyperparameters 517 to generate an output. The output may be within the predefined threshold of the target output used during the training process. The output may be the predicted total signal intensity R_{predicted} of a probe signal for raw or normalized value (e.g., Norm R_{predicted}, ENorm R_{predicted}, or LRR_{predicted}). Though illustration and description may relate to particular types of machine learning models, such as a linear regression model, a random forest model, or a neural network, the parameters 517 (e.g., weights, biases, coefficients, etc.) of other types of machine learning models 509 may similarly be trained and/or implemented, as described herein.

A number of different probe features 503a have been considered and may be defined as input 503 for each machine learning model 509. For example, the probe features 503a may include a primer melting temperature (TM) under one or more salt concentrations. The following TABLE 2 comprises a set of primer TM that were computed using the primer3 package. See Koressaar T, Lepamets M, Kaplinski L, Raime K, Andreson R and Remm M. Primer3_masker, Integrating Masking of Template Sequence with Primer Design Software, Bioinformatics 2018; Volume 34, Issue 11:1937-1938.

TABLE 2

| Parameter | | |
|---|---|---|
| configuration | mono | di |
| 1 | 50 | 0 |
| 2 | 50 | 1 |
| 3 | 50 | 2 |
| 4 | 50 | 3 |
| 5 | 25 | 0 |
| 6 | 75 | 0 |
| 7 | 100 | 0 |

Each parameter configuration may be a different probe feature 503a that is included as an input 503 into the machine learning model 509.

The probe features 503a may be defined by an amount of GC content in a target region of the probe. For example, different probe features 503a may be defined based on the GC ratio or GC content within a proportion of the probe. In an example, a first probe feature may include a GC proportion within 10 kb of the probe and a second probe feature may include a GC proportion within 100 kb of the probe.

The probe features 503a may be defined by a gene/pseudogene count intersecting a target. For example, a probe feature may be defined as: a number of genes intersecting a 50 bp probe; a number of genes within a 10 kb of the probe: a number of genes within 100 kb of the probe; a number of genes within 1 mb of the probe; a number of pseudogenes intersecting a 50 bp probe; a number of pseudogenes within a 10 kb of the probe; a number of pseudogenes within 100 kb of the probe; and/or a pseudogenes of genes within 1 mb of the probe.

The probe features 503a may be defined by an intersection of a target region with repeat categories. In one example, probe features may be defined by 20 Boolean features representing whether the 50 bp probe intersected the repeat or not. The probe features may be defined by 20 repeat categories obtained by RepeatMasker track from UCSC. One example of the repeat categories is provided in the following article by Jurka J. Repbase, entitled “Update: a database and an electronic journal of repetitive elements,” Trends Genet. 2000 Sep. 16(9):418-420. PMID: 10973072. Another example of the repeat categories is shown at the following web address: https://genome.ucsc.edu/cgi-bin/hgTrackUi?g=rmsk, entitled “Repeating Elements by RepeatMasker,” last updated Sep. 3, 2021. The probe features may be defined by a count of frequency of k-mers. Additionally, or alternatively, the probe features may be defined by an entropy of the k-mers.

The probe features 503a may be defined by a DNase signal in a target. For example, the probe features may be defined by a mean DNAase signal of each of the Roadmap Epigenomics cell types. See e.g., Roadmap Epigenomics Consortium., Kundaje, A., Meuleman, W. et al. Integrative analysis of 111 reference human epigenomes. Nature 518, 317-330 (2015). Additionally, or alternatively, the probe features may be defined by the following cell type-specific DNA signals selected to represent a range of cell types: E096: Lung; E066: Liver; E065: Aorta; E071: Brain Hippocampus Middle; E030: Primary neutrophils from peripheral blood; E046: Primary natural killer cells from peripheral blood; E032: Primary B cells from peripheral blood; E063: Adipose nuclei; E108: Skeletal muscle female; and/or E107: Skeletal muscle male.

The probe features 503a may be defined by a homologous region count. For example, the homologous region count may be the number of homologous regions based on GENCODE parent-pseudogene annotation. See e.g., Frankish A, el al. GENCODE reference annotation for the human and mouse genomes. Nucleic Acids Res. 2019 Jan. 8; 47(D1):D766-D773. Doi: 10.1093/nar/gky955. PMID: 30357393; PMCID: PMC6323946.

Each machine learning model 509 may receive an entire predefined set of probe features 503a or a subset of probe features 503a as input 503. The subset of probe features 503a may include the probe features for a predefined k-length substring (k-mer) of the probes. The k-mer features may be fewer probe features than may be included in the entire predefined set, which may require less processing for the machine learning model 509 and may take less time to train. Simpler machine learning models, such as the linear regression model or the random forest model, may receive the k-mer features as input. The larger set of predefined features may take more processing and may take more time to train the machine learning model 509. As such, the larger set of predefined features may be input into more complex models, such as a neural network or a random forest model. The more complex machine learning models may perform better than the simpler machine learning models, but may take more time and/or computing resources to train and/or implement. Additionally, the random forest model may perform better using a subset of probe features as input, since it does not explicitly model spatial dependencies. However, random forest may perform well for interpretation of feature importance and/or feature selection.

In an example, a linear regression model or a random forest model may receive an input 503 of the one or more probe features 503a. The probe features 503a may include probe sequence features (e.g., kmers, entropy, and/or one-hot encoding) and/or genomic context features (e.g., other features). As an example, the linear regression model or the random forest model may receive as input k-mer features for k-mers having a count of k:=1-3 in a probe. The k-mer features may include up to 84 features. In another example, the number of features evaluated may be reduced by discarding k-mer features having low entropy. In this example, the linear regression model or the random forest model may receive in input of k-mer features for k-mers having a count of k=1-4 in a probe. The k-mer features may include 4 features.

FIG. 5B shows an example architecture of a neural network 500. For example, as shown in FIG. 5B, a neural network 500 may have multiple input layers 502, 504 for receiving an input for a convolutional portion of the neural network 500 and a fully-connected feed forward portion of the neural network 500, respectively. Convolutional neural networks have been successfully utilized in many genomics applications. However, most applications use DNA sequences as a single input to the model.

The neural network 500 is a hybrid neural network architecture combining convolutional layers of a convolutional neural network and dense layers of a fully-connected feedforward neural network. A difference between a densely connected layers and convolution layers is that dense layers learn global patterns in their input feature space, whereas convolution layers learn local patterns found in a convolutional filter applied to the inputs. As a result, the convolutional portion of the neural network 500 may learn patterns that are translation invariant and it may learn spatial hierarchies of patterns. This allows convolutional neural networks to efficiently learn increasingly complex and abstract visual concepts.

A convolutional neural network learns highly nonlinear mappings by interconnecting layers of artificial neurons arranged in many different layers with activation functions that make the layers dependent. It includes one or more convolutional layers, interspersed with one or more subsampling layers and non-linear layers, which are typically followed by one or more fully connected layers. Each element of the convolutional neural network receives inputs from a set of features in the previous layer. The convolutional neural network learns concurrently because the neurons in the same feature map have identical weights. These local shared weights reduce the complexity of the network such that when multi-dimensional input data enter the network, the convolutional neural network avoids the complexity of data reconstruction in the feature extraction and regression or classification process.

As shown in FIG. 5B, the neural network 500 may have input layers 502, 504 for receiving input for the convolutional portion of the neural network and the fully-connected feed forward portion of the neural network, respectively. As described herein, the neural network may receive the probe features 503a at the input layer 504. In the provided example, the input layer 504 may receive an array of 48 probe features 503a. However, the number of probe features 503a may vary. The probe features 503a may be passed from the input layer to a dense layer that performs a non-linear transformation using a ReLU function with a 50% dropout. The output of the dense layer is an array having a height of 32 and a width of 1.

Though other machine learning models described herein may also receive probe features 503a as input, the neural network 500 may receive another input at the input layer 502. The input layer 502 may receive a probe sequence 503b. The probe sequence 503b may be received as a one-hot encoded 50 bp probe sequence. The input may be passed through convolution layers which perform a convolution operation between the input values and convolution filters (matrix of weights) that are learned over many gradient update iterations during training. A convolution operation works by sliding the filters having a defined kernel size over an input feature map (also referred to as a 3D tensor) according to the stride, and extracting the patch of surrounding features. Each such patch is then transformed (via a tensor product with the same learned weight matrix, called the convolution kernel) into an ID vector of shape (output depth). Each of these vectors are then spatially reassembled into a 3D output map of shape (height, width, output depth).

In the example neural network 500 provided in FIG. 5B, the input of the probe sequence 503b is provided as a feature map having a depth of 4, a height of 50, and a width of 1. The depth may correspond to the number of standard nucleotides (e.g., A, C, T, G). This one-hot encoding may be equivalent to encoding each nucleotide into a one-by-four vector and concatenating them. The input is passed through a first hidden convolutional layer that comprises 32 filters, each with a kernel size of 3×3, a stride of 1, and a padding of 1, followed by a ReLU activation function. The output feature map from the first convolutional layer has a depth of 32, a height of 50, and a width of 1. This feature map is passed through a second hidden convolutional layer that comprises 8 filters, each with a kernel size of 3×3, a stride of 1, and a padding of 1, followed by a ReLU activation function. The output feature map from the second convolutional layer has a depth of 8, a height of 50, and a width of 1. This feature map is passed through a third hidden convolutional layer that comprises 16 filters, each with a kernel size of 5×5, a stride of 1, and a padding of 2, followed by a ReLU activation function. The output feature map from the third convolutional layer has a depth of 16, a height of 50, and a width of 1.

The output of the third convolutional layer is concatenated by appending each of the 16 columns to generate an array having a height of 800 and a width of 1. The array is passed through a dense layer that performs a non-linear transformation using a ReLU function with a 50% dropout. The output of the dense layer may include an array having a height of 128 and a width of 1. The output of the dense layer may be concatenated.

The output from the convolutional portion of the neural network 500 may meet the output from the feedforward portion of the neural network 500 at a junction for performing non-linear transformations. The output from the convolutional portion of the neural network 500 and output from the feedforward portion of the neural network 500 are passed through a dense layer that performs a non-linear transformation using a ReLU function with a 50% dropout. The combined output is passed through another dense layer that provides predicted total signal intensity R_{predicted} 515 of a probe signal for each of the 50 base-pair input into the input layer 502.

The convolutional portion of the neural network 500 captures the probe target sequence (e.g., 50 bp in this case), while the feedforward portion of the neural network 500 captures and integrates large scale genomic signatures. Genetic features and epigenetic state surround the target region affect the probe signal but may not be effectively captured by a traditional convolutional neural network due to the sequence length and complexity. We generated 48 additional features that summarize the genetic and epigenetic data for up to 1 MB from the target region. With the hybrid network architecture of the neural network 500, local and global sequence and epigenetic features of diverse nature are effectively incorporate into the machine learning model.

Training the convolutional portion of the neural network 500 may allow the convolutional portion of the neural network 500 to capture different patterns in the probe sequence 503b (e.g., 50 bp in this case). The patterns that are capable of being detected by the convolutional portion of the network may include GC content (e.g., proportion of G bases and C bases in the 50 bp probe sequence). The convolutional portion of the neural network 500 may capture the shape of the DNA and predict how likely the DNA is to bind based on the characteristics of the DNA sequence. Training the feedforward portion of the neural network 500 may allow the feedforward portion of the neural network 500 to more accurately predict the predicted total signal intensity R_{predicted} 515 of the probe signal for each of the 50 base-pair input into the input layer 502.

Each of the machine learning models may include parameters (e.g., weights and/or biases) that may be trained based on the sample-specific image data of the probe signal received from the genotyping device. In these sample-specific models, each machine learning model may be trained for each individual using the probes as training samples. During training, the probes with no signal data in the image data may be removed and the remaining signal data from the image data may be split the data into training data, test data, and/or validation data, as further described herein.

One example training framework for the linear regression model, the random forest model, and/or the neural network may include holding out 10% or 20% of the sample-specific image data of the probes for testing. The remaining 90% or 80% may be used as training data.

The random forest model and/or the neural network may further comprise hyperparameters, which may be the variables that govern the training process itself. For example, the hyperparameters for the random forest model may include the maximum depth of the trees (“max_depth”) and the number of trees used (“n_estimators”). The hyperparameters of the neural network may include the number of hidden layers of nodes to use between the input and output layers, the number of nodes each hidden layer should use, batch size, and epochs.

These variables are not directly related to the training data but are configuration variables. The parameters may change during training, while hyperparameters may remain constant during a training session using the training data. The random forest model and the neural network hyperparameters may additionally be tuned and have been tuned to predict the response variable as output, as described herein.

In one example, we tested the max, depth values of 5, 10, 20, and n estimator values of 50, 100, 200. We performed this grid search on a single randomly chosen sample. Example hyperparameters for each response variable are provided in TABLE 3 below.

TABLE 3

| Response Variable | max_depth | n_estimators |
|---|---|---|
| Norm R | 20 | 200 |
| Raw R | 20 | 200 |
| ENorm R | 20 | 200 |
| LRR | 10 | 200 |

The random forest model may implement N-fold cross validation for hyperparameter selection. In one example, the random forest model may implement a 3-fold cross validation for hyperparameter selection.

When implementing a neural network, the hyperparameters may be tuned. For example, different numbers of hidden layers and nodes have been implemented. An example of the number of hidden layers and nodes is provided in FIG. 5B and the description herein. However, the convolutional layers may have different numbers of filters (e.g., 16, 32, 64, etc.) and/or a different kernel size (e.g., 3×3 or 5×5). The dense layers may also have a different number of hidden nodes (e.g., 16, 32, 64, 128, 256, 512, etc.). When validating the neural network, 10% of the sample-specific image data of the probes may be held out as validation data for hyperparameter selection and early stopping. In an example, the neural network having the structure illustrated in FIG. 5B has been trained with a batch size of 128, with 60 epochs (early stopping with patience=5), a means-squared error loss function, and an initial learning rate of 1 e⁻⁴. Alternative batch sizes (e.g., 32, 64, 128, 256, etc.) and/or epochs (e.g., 10, 20, 30, 40, 50, 60, 70, 80, 90, 100, etc.) may be implemented. The neural network may be trained using the Adam optimization algorithm for error correction and to update the weights and biases based on the training data. The neural network may be trained on a single GPU, though may also or alternatively be trained with CPU.

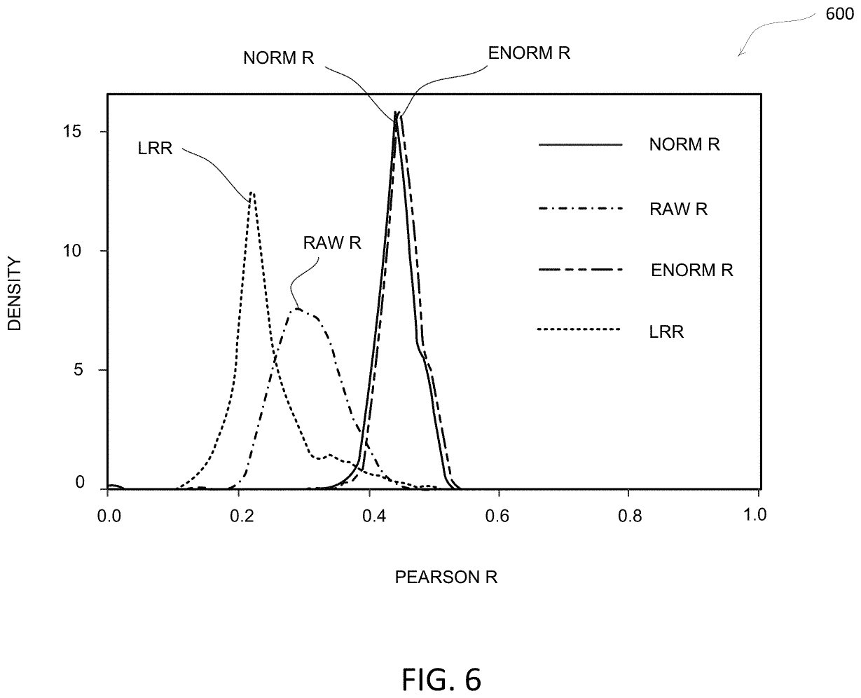

The prediction accuracy of the predicted total signal intensity R_{predicted} of a probe signal using different response variables (e.g., Norm R, Raw R, ENorm R, and LRR) as the measures of total signal intensity of a probe R and different machine learning models has been tested. FIG. 6 includes a graph 600 that illustrates an example of the prediction accuracy of the different measures of the total signal intensity of the probe using the linear regression model. The model was trained separately for 1335 individuals. The graph 600 illustrates the prediction accuracy calculated as a Pearson correlation coefficient between the predicted total signal intensity R_{predicted} of a probe signal and the observed total signal intensity R of the probe signal. The graph 600 illustrates the Pearson correlation on the x-axis, as computed between the observed and predicted values of the held out test data for the linear regression model. As shown in the graph 600, ENorm had the highest prediction accuracy, followed by Norm R. Similar comparisons of the prediction accuracy were performed for the random forest model and the neural network. For each of the linear regression model, the random forest model, and the neural network, the ENorm R response variable had the highest prediction accuracy, followed by Norm R.

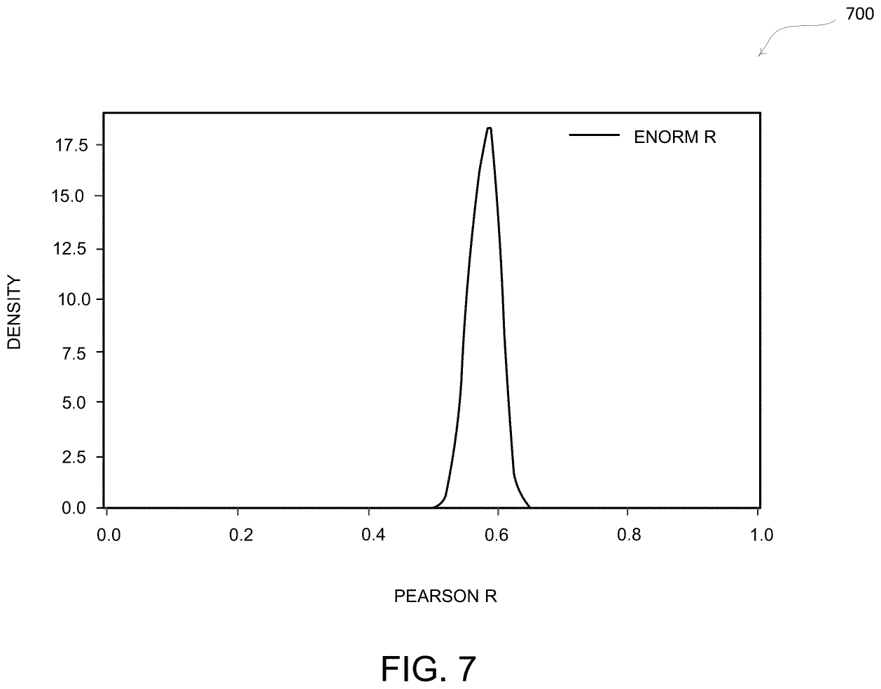

FIG. 7 includes a graph 700 that illustrates an example of the prediction accuracy of the total signal intensity of the probe using the random forest model being trained for predicting the ENorm R as the response variable. The graph 700 illustrates the prediction accuracy calculated as a Pearson correlation coefficient between the predicted total signal intensity R_{predicted} of a probe signal and the observed total signal intensity R of the probe signal. The graph 700 illustrates the Pearson correlation on the x-axis, as computed between the observed and predicted values of the held out test data for the random forest model. When comparing the prediction accuracy of ENorm R as shown in the graph 700 with the ENorm R as shown in the graph 600 in FIG. 6, the prediction accuracy was higher for the random forest model than for the linear regression model.

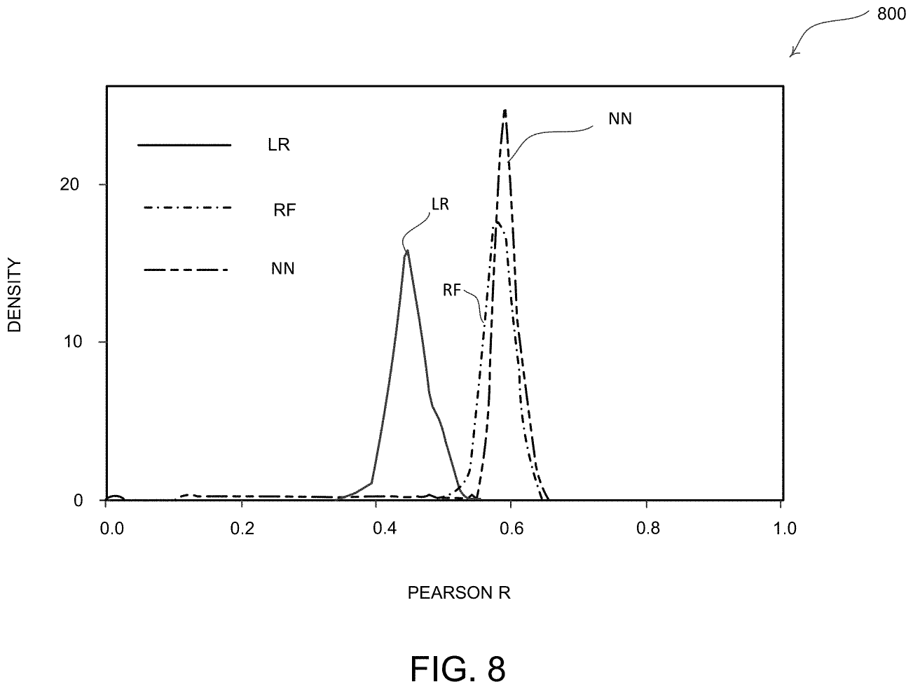

The prediction accuracy of each of the machine learning models was also tested for predicting the accuracy of each of the different response variables (e.g., Norm R, Raw R, ENorm R, and LRR). FIG. 8 includes a graph 800 that illustrates an example of the prediction accuracy of the different machine learning models. The machine learning models were trained separately for 1335 individuals. The graph 800 illustrates the prediction accuracy calculated as a Pearson correlation coefficient between the predicted total signal intensity R_{predicted} of a probe signal and the observed total signal intensity R of the probe signal. The graph 800 compares the Pearson correlation on the x-axis, as computed between observed and predicted values of the held out test data for each of the machine teaming models. As shown in the graph 800, the neural network performed the best on average, followed by the random forest model.

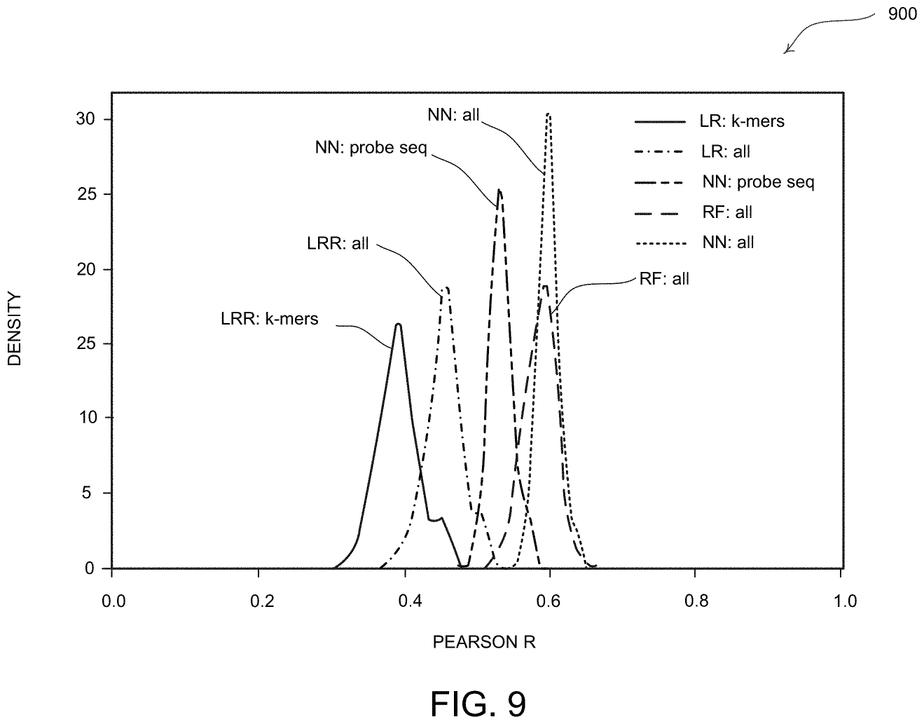

FIG. 9 includes a graph 900 that illustrates an example of the prediction accuracy of different machine learning models accepting different types of input for predicting the same response variable (e.g., Norm R, Raw R, ENorm R, or LRR). The graph 900 illustrates the prediction accuracy calculated as a Pearson correlation coefficient between the predicted total signal intensity R Mica of a probe signal and the observed total signal intensity R of the probe signal. The graph 900 compares the Pearson correlation on the x-axis, as computed between observed and predicted values of the held out test data for each of the machine learning models. As shown in the graph 900, the neural network that receives each of the probe features as input (e.g., NN: all) performs the best on average, followed by the random forest model that receives each of the probe features as input (e.g., RF all) performs similarly. The neural network that receives the probe sequence (e.g., 50 bp) as input (e.g., NN: probe seq), but does not receive the additional probe features as input performs relatively better than the linear regression models. This neural network may be a convolutional neural network without the dense layers for the annotation data input. The linear regression model that receives each of the probe features as input (e.g., LR all) performs relatively better than the linear regression model that receives k-mer features, or a subset of the features. The neural network that receives the probe sequence (e.g., 50 bp) as input (e.g., NN: probe seq), but does not receive the additional probe features as input may be trained similarly to the hybrid neural network that receives the additional probe features.

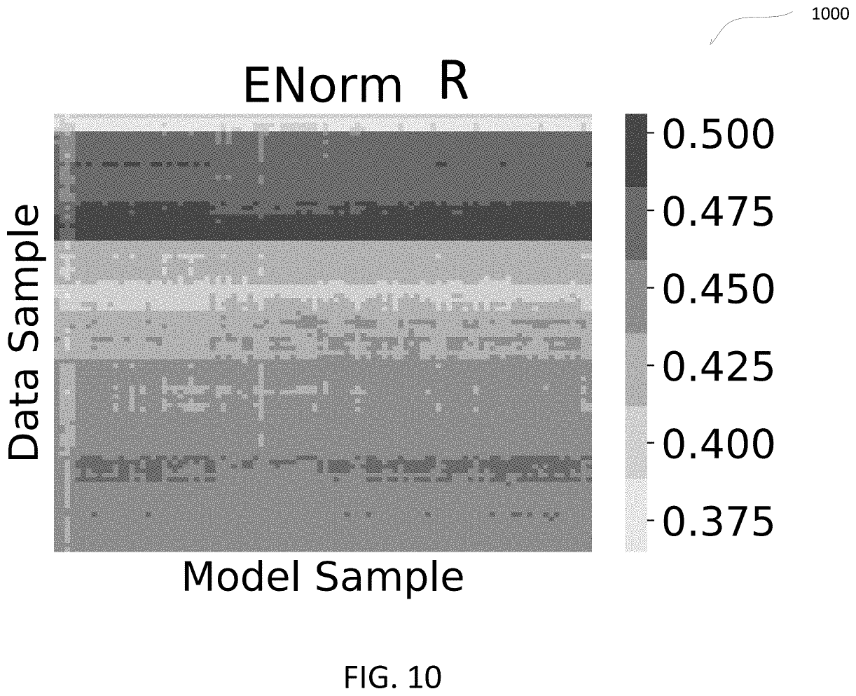

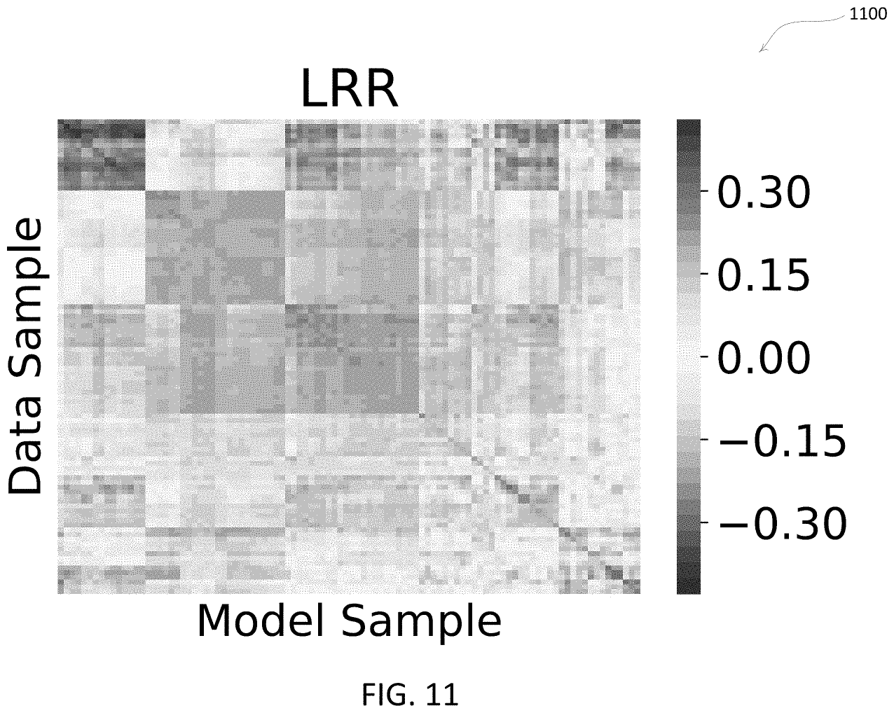

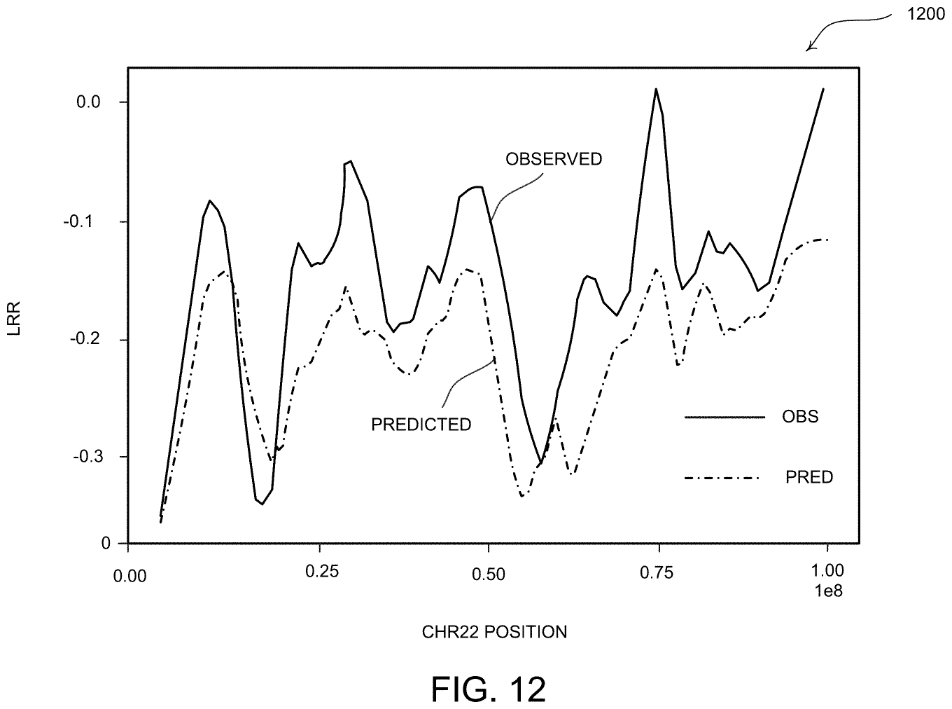

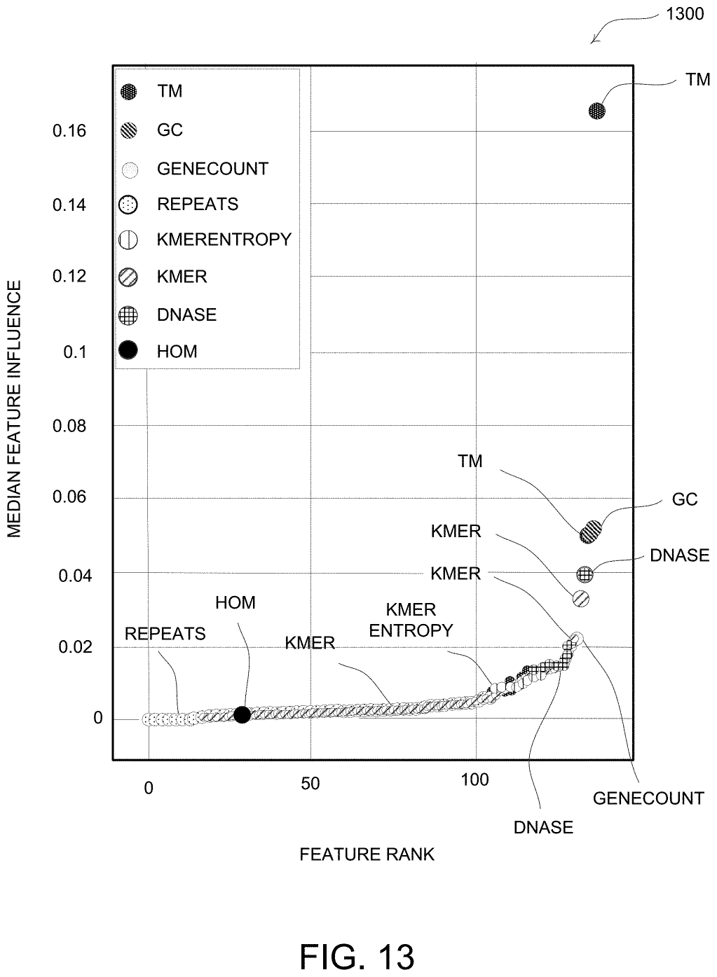

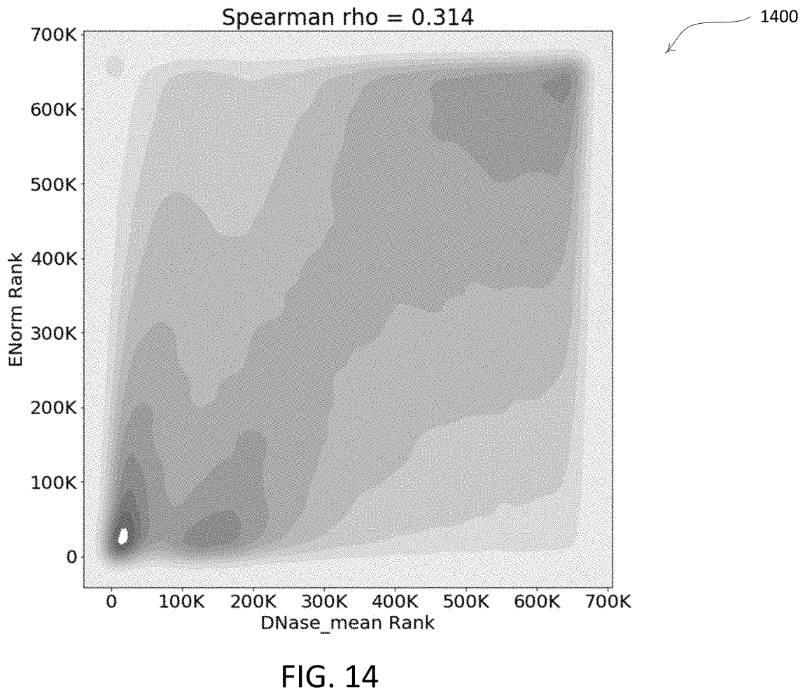

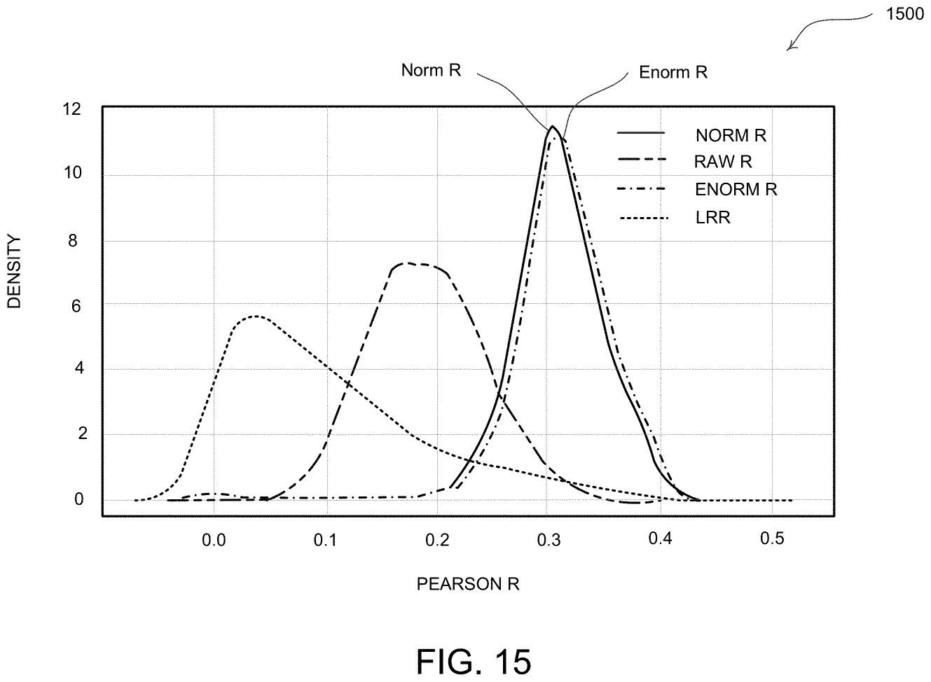

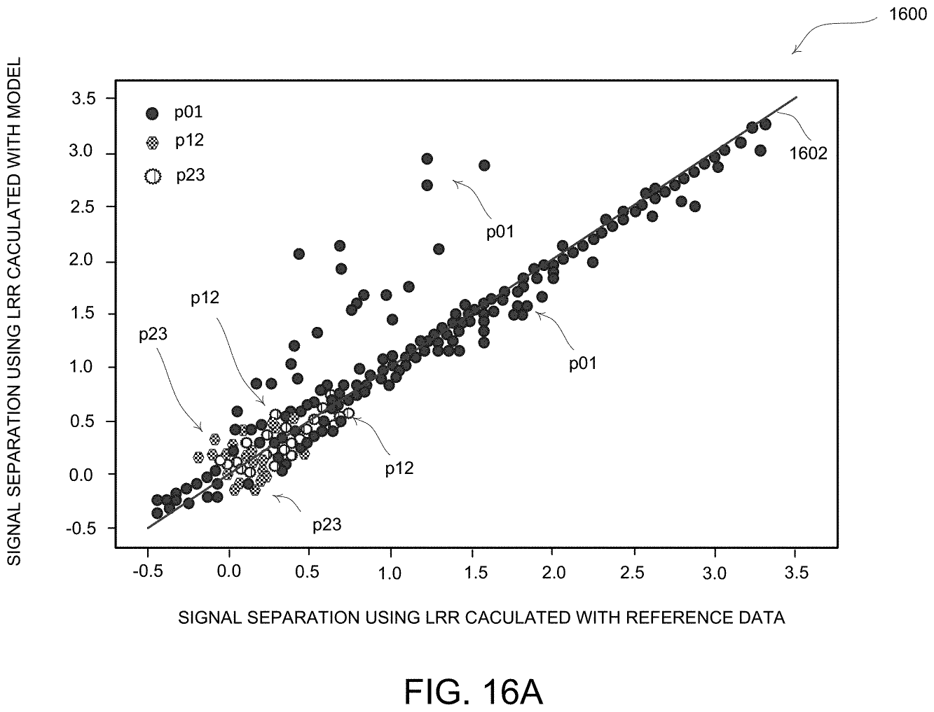

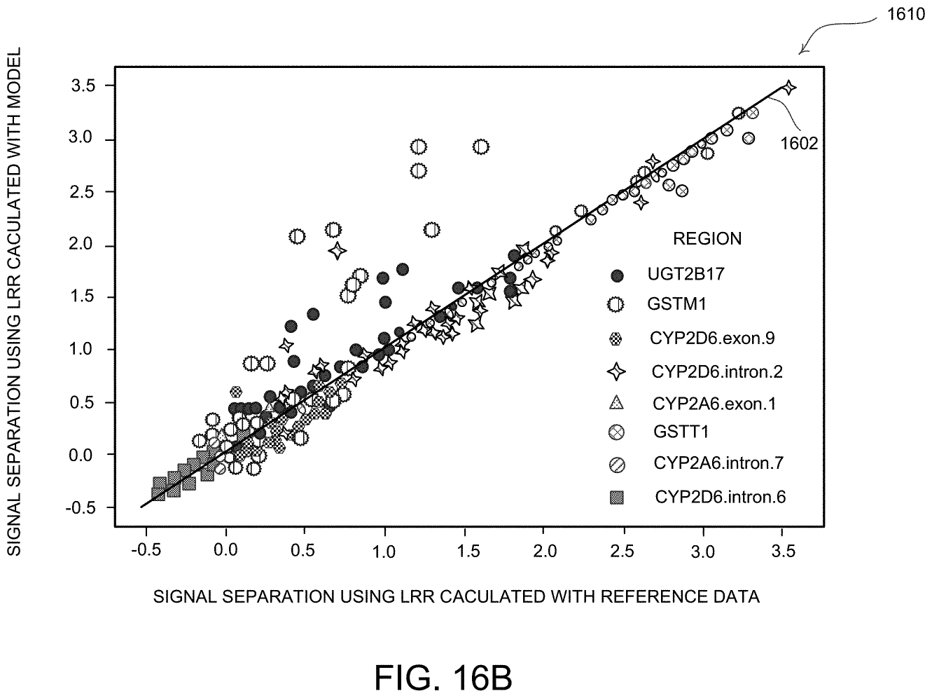

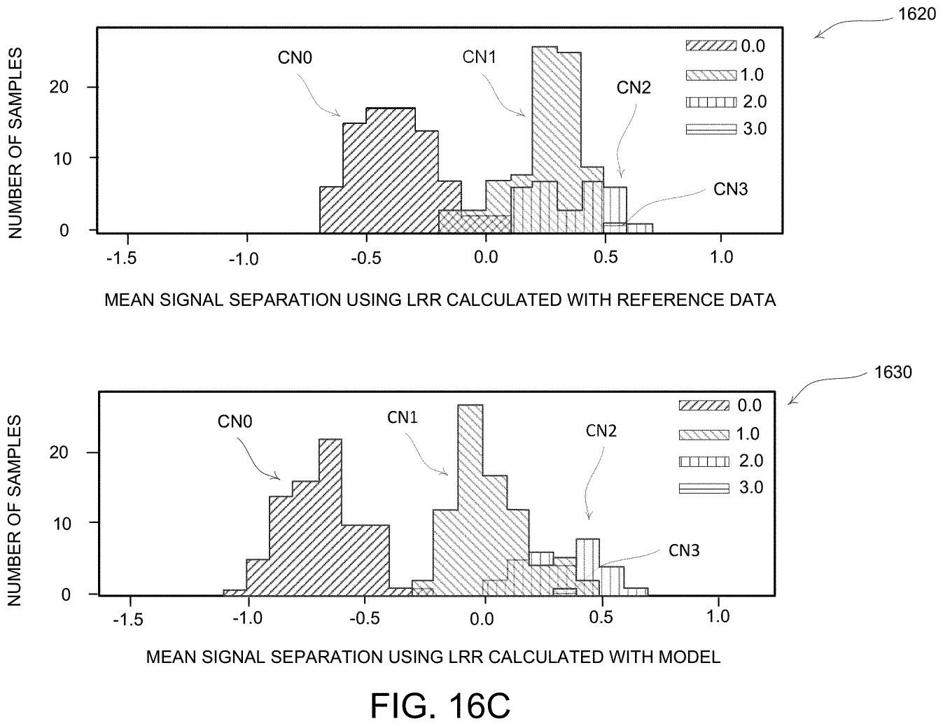

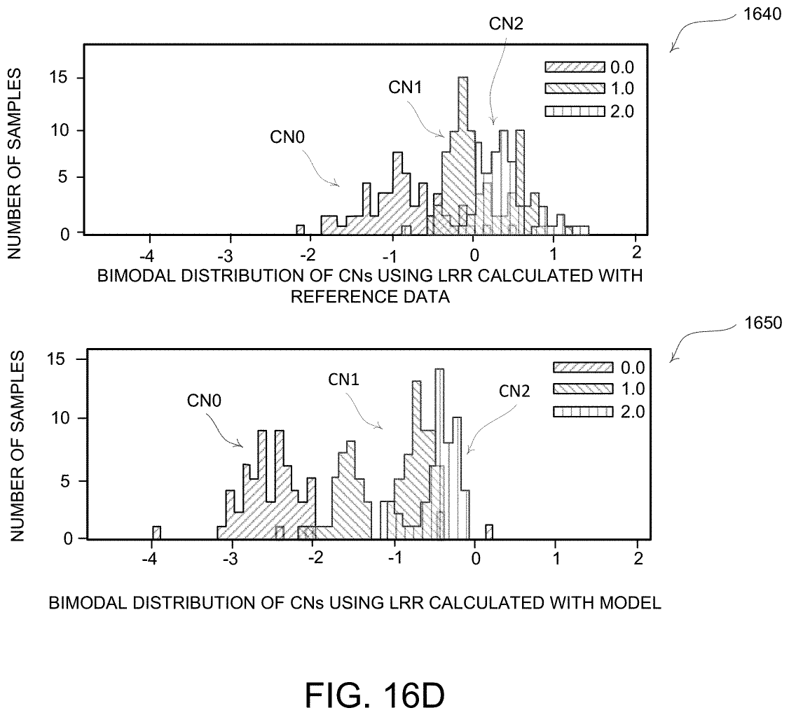

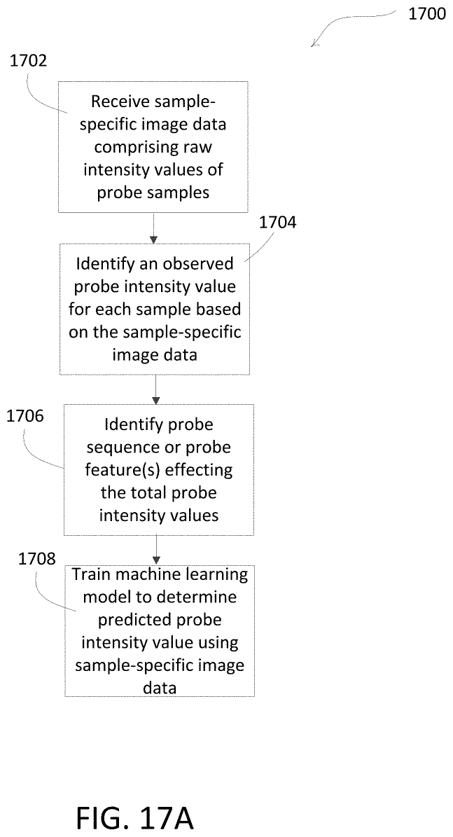

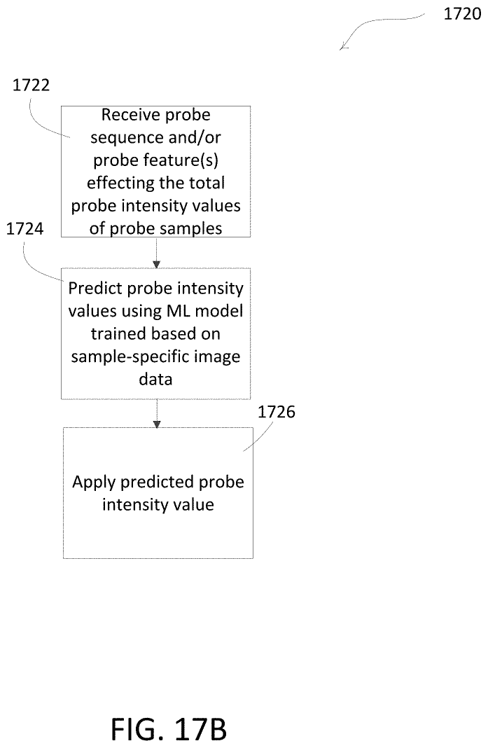

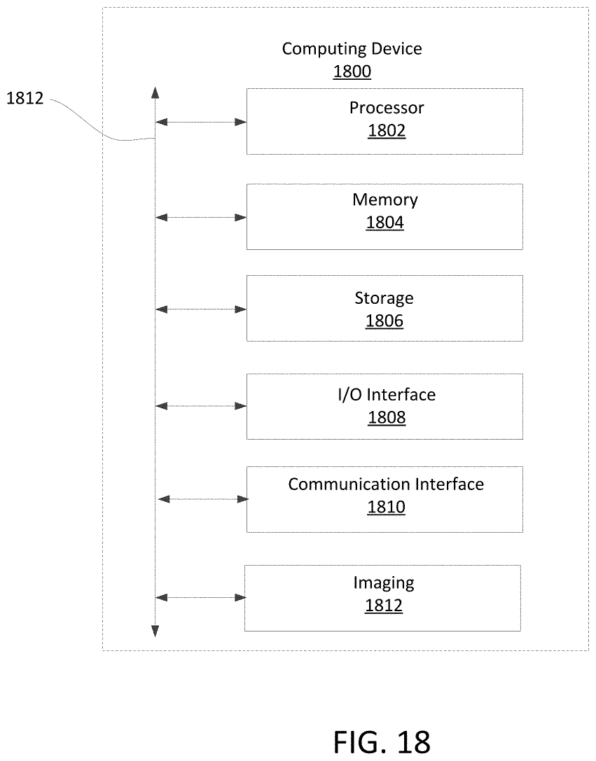

The generalization of each of the machine learning models across samples was also tested. FIG. 10 includes a heatmap 1000 that shows a Pearson correlation of observed and predicted values of the test data for each combination of models on the x-axis and the data set on the y-axis. The ordering of the samples is the same for each axis. The heatmap 1000 illustrates an example of how well each of the machine learning models that were trained for predicting ENorm R generalize across samples. For 100 samples, each sample-specific model was applied to the test data from each of the other samples. In the heatmap 1000, the columns correspond to the sample-specific model and the rows correspond to the sample data used. The ordering of the samples in the rows and columns is the same. The color or shading corresponds to the Pearson correlation of the true and predicted values. As shown in FIG. 10, the machine learning models that were trained for predicting ENorm R were very similar across individuals.

Due to the high generalization across samples for the linear model, a single neural network was trained using the mean across samples and applying it to all samples. The sample-specific neural network performed slightly better than the neural network trained using the mean across samples. This indicates that a model trained using the mean may be used as an accurate approximation for the expected signal intensity. Additionally, by training the single model, training time may be reduced.

Certain response variables (e.g., Norm R, Raw R, ENorm R, and LRR) may generalize well across samples, while other response variables may be more sample-specific. A similar test was performed for predicting how LRR generalizes across samples. As shown in FIG. 11, different machine learning models may be observed between individuals. FIG. 11 includes another heatmap 1100 that shows a Pearson correlation of observed and predicted values of the test data for each combination of models on the x-axis and the data set on the y-axis. The ordering of the samples is the same for each axis. The heatmap 1100 illustrates an example of how well each of the machine teaming models that were trained for predicting LRR generalize across samples. For 100 samples, each sample-specific model was applied to the test data from each of the other samples. In the heatmap 1100, the columns again correspond to the sample-specific model and the rows again correspond to the sample data used. The color or shading corresponds to the Pearson correlation of the true and predicted values. The ordering of the samples in the rows and columns is the same.

In the heatmap 1100, the strong diagonal indicates that each sample data is best predicted by the model that was trained using that data. Additionally, some individuals are very similar, while others are anticorrelated. These differences correspond to differences in the genomic wave, as described herein.

The predicted total signal intensity R_{predicted} for LRR machine learning models should vary across individuals, similarly to the observed and expected total signal intensity of a sample. FIG. 12 includes a graph 1200 that illustrates an example of the observed total signal intensity R and the predicted total signal intensity R_{predicted} of a probe signal. The observed total signal intensity value is an LRR value and the predicted total signal intensity R_{predicted} value is a predicted LRR value. As shown in FIG. 12, the predicted LRR values track the genomic waves of the observed total signal intensity value LRR.

In addition to the accuracy of the machine teaming models, the influence of each the probe features on the predicted total signal intensity Rₚea has been tested. FIG. 13 includes a graph 1300 that illustrates an example of a feature rank and a median feature influence on a random forest model for each of eight probe features that were tested. The k-mer probe features were input into a random forest model that is trained using the normalized total intensity ENorm R, such that the predicted total signal intensity R_{predicted} is predicting an ENorm value. As shown in the graph 1300, TM and GC content have a relatively higher influence on the predicted total signal intensity Ra. Another feature having a relatively higher influence on the predicted total signal intensity R_{predicted} is the amount of DNase signal in the target region of the probes. As described herein, DNase measures the DNA accessibility of a region. When compared to the feature importance in linear regression models, the same k-mer features contributed more to the random forest model. Non-linear interactions may capture some effect of repeat elements and/or TM features on the predicted total signal intensity R_{predicted}. Position-specific sequence information may be incorporated when using a more complex model, such as a neural network. By giving a whole probe sequence as input, the model is given the information contained in the k-mer features and additional information about the position of the k-mers relative to each other. This may allow the model to identify the effect of relative position of the k-mers to total signal intensity.

FIG. 14 includes a heatmap 1400 that illustrates an example of spearman correlation of DNase mean rank with the normalized total intensity ENorm R. As described herein, DNase is a measure of the DNA accessibility of the region. The spearman correlation of DNase with the total probe signal intensity R is 0.3, which indicates a relatively higher influence than other features, including GC content.

Since TM is a probe feature that was the most influential in predicting the total signal intensity R_{predicted}, machine learning models were trained using TM as a single probe feature as input. FIG. 1S includes a graph 1500 that illustrates an example of the prediction accuracy of the different measures of the total signal intensity of the probe using the linear regression model that has been trained using TM as a single probe feature as input. The model was trained separately for 1335 individuals. The graph 1500 illustrates the prediction accuracy calculated as a Pearson correlation coefficient between the predicted total signal intensity R_{predicted} of a probe signal and the observed total signal intensity R of the probe signal. When compared to the graph 600 shown in FIG. 6, the prediction accuracy indicates that the machine learning model trained using TM as a single probe feature as input may be implemented.

FIG. 16A includes a graphical illustration 1600 showing examples of signal separation for total signal intensity calculated using different embodiments described herein. As described herein, a greater signal separation between signals may increase the accuracy of CNV calling. In generating the data illustrated in FIG. 16A, the normalized signal intensity Norm R_{expected} and the normalized predicted total signal intensity R_{predicted} was used respectively in computing LRR from Norm R. The normalized signal intensity Norm R_{expected} may be used to calculate LRR (e.g., using Equation 3 herein) to identify the signal variation between copy numbers for given individuals. As described herein, LRR may be used to predict a copy number for a given individually and LRR may be difficult to compute using Norm R_{expected}, due to the reliance on a reference dataset as described herein.

The signal plot on the x-axis of the graphical illustration 1600 illustrates a signal separation for LRR calculated using Norm R_{expected}, which has been calculated based on a reference dataset as described herein. In contrast, the signal plot on the y-axis of the graphical illustration 1600 illustrates a signal separation for LRR calculated using Norm R_{predicted} based on the machine learning model implementing addNorm, described herein.

As shown in the graphical illustration 1600 in FIG. 16A, there are three different types of plots (p01, p12, and p23). Each of the plots correspond to an average distance in signal between different copy numbers. Plot p01 is the average difference between copy number 1 (CN1) and copy number 0 (CN0). Plot p12 is the average difference between copy number 2 (CN2) and copy number 1 (CN1). Plot p23 is the average difference between copy number 2 (CN2) and copy number 3 (CN3). LRR should be different between people with different copy numbers. CNV calls may be made by comparing LRR values of a sample with LRR values of samples with known copy numbers. The greater the distance between copy numbers, the greater the power for differentiating copy number between different people. As shown in the graphical illustration 1600 in FIG. 16A, the plots above the line 1602 show a greater separation between the signals for each copy number than the separation of the plots below the line 1602, which indicates a greater separation in the signals for using the normalized signal intensity calculated using the model than the normalized signal intensity calculated using the reference dataset.

Different sample-specific probe intensity values may vary from the probe intensity values of reference datasets independent of copy number variation, as the amplitude and phase of the signals may be sample-specific. As a result, the use of sample-specific image data and the models described herein when generating the normalized total signal intensity may be more accurate than the use of the reference datasets due to the biases introduced by the generation of the reference dataset described herein.

FIG. 16B includes a graphical illustration 1610 showing similar examples of signal separation for different genes or regions of probes of particular interest in a genome. In generating the data illustrated in FIG. 16B, the normalized signal intensity Norm R_{expected} and the normalized predicted total signal intensity R_{expected} was similarly used to compute LRR from Norm R. The plots above the line 1602 show a greater separation between the signals for each copy number than the separation of the plots below the line 1602, which indicates a greater separation in the signals for each region of the genome using the normalized signal intensity calculated using the model than the normalized signal intensity calculated using the reference dataset.

FIG. 16C includes two graphical illustrations 1620, 1630 showing a mean signal separation of a particular probe in which the signal separation using the LRR calculated with a model was greater than signal separation computed using reference data. Each histogram includes a distribution of LRR across multiple samples. p01, as shown in FIG. 16A is computed as a mean of CN1 minus a mean of CN0.

The graphical illustration 1620 shows the mean signal separation for each copy number when the normalized signal intensity calculated using the reference dataset. The graphical illustration 1630 shows the difference in the mean signal separation for each copy number (e.g., CN0, CN1, CN2, and CN3) when the normalized signal intensity is calculated using the models described herein. The x-axis in each of the graphical illustrations 1620, 1630 illustrates the relative amount of signal separation between each of the copy numbers. As shown in the graphical illustrations 1620, 1630 of FIG. 16C, the signal separation is greater when the normalized signal intensities are calculated using the model than when the normalized signal intensities are calculated using the reference dataset. This greater amount of separation will allow for more accurate CNV calling.

FIG. 16D includes two graphical illustrations 1640, 1650 showing bimodal distributions for each of the different copy numbers illustrated by the plots in FIG. 16A. The bimodal distributions may indicate poorly normalized data, which can adversely affect CNV calling. The graphical illustration 1640 shows the bimodal distribution for each copy number when the normalized signal intensity is calculated using the reference dataset. The graphical illustration 1650 shows the bimodal distribution for each copy number when the normalized signal intensity is calculated using the models described herein. As shown in the graphical illustrations 1640, 1650 of FIG. 16C, the bimodal distribution is improved for some copy numbers when the normalized signal intensities are calculated using the model as described herein. The different bimodal distributions in the graphical illustration 1640 may indicate the normalized signal intensity Norm R_{expected} that relies on the reference dataset may be inaccurate. When the normalized signal intensity Norm R_{predicted} is calculated using the models described herein, some of the bias in the reference data set may be removed to allow for a more accurate normalized signal intensity. The use of the model may prevent overfitting that may occur with the use of the reference dataset. Each dataset is a bit different and each sample is a bit different, so the use of the sample-specific data to train the model and then use the same sample data set for implementation and generating the normalized signal intensity Norm R_{predicted} may allow for a more accurate normalized value.

FIG. 17A is a flowchart depicting an example procedure 1700 for training a machine learning model to predict the total signal intensity R_{predicted} of a probe signal. The one or more portions of the procedure 1700 may be performed by one or more computing devices. The one or more computing devices may reside on or be external to a genotyping device. One or more portions of the procedure 1700 may be stored in memory as computer-readable or machine-readable instructions that may be executed by a processor of the one or more computing devices. Though portions of the procedure 1700 may be described herein as being performed by a single computing device, the procedure 1700, or portions thereof, may be distributed across multiple devices, such as a client computing device, a genotyping device, and/or one or more server computing devices.

The procedure 1700 may begin at 1702. As shown in FIG. 17A, at 1702 the computing device may receive sample-specific image data. The computing device may also, or alternatively, receive an average value of each of the samples to perform a similar procedure 1700 using the average values. For example, the average value may be an ENorm or R Norm value. The sample-specific image data may be associated with a sample relating to a single individual. The sample-specific image data may include raw x and y signals that represent raw probe intensity values of different colored signals (e.g., red and green signals) of sections of the image-generating chip that are generated in response to the fluorescent labels for genotypes A and B. The sample-specific image data may be received at 1702 by the computing device from an external genotyping device or by portions of a computing device within a genotyping device capable of generating the image data.

At 1704, the computing device may identify an observed probe intensity value R for the sample based on the sample-specific image data. The total probe intensity value may be a total raw probe intensity value R (e.g., Raw R) or a total normalized probe intensity value (e.g., Norm R, ENorm R, or LRR) may be calculated from the raw probe intensity value R, as described herein.

At 1706, the computing device may identify a probe sequence or one or more probe features effecting the probe intensity values. For example, as further described herein, the probe features may include an entire set of predefined probe features or a subset of probe features. The probe features may include probe sequence features (e.g., kmers, entropy, and/or one-hot encoding) and/or genomic context features (e.g., other features). Though genomic context features may be described, these probe features may also be referred to as annotation features, as these features may be derived from external annotations of the genome/epigenome. The subset of probe features may be k-mer features for a k-mer of a probe sequence. The probe sequence may include an entire probe sequence or a portion thereof. For example, the probe sequence may include a variety of lengths within the entire probe sequence or the entire probe sequence. Different machine learning models may be configured with different input layers for inputting an entire probe sequence (e.g., 50 bp probe sequence), the entire set of predefined probe features, or a subset of probe features.

At 1708, the machine learning model may be trained, using sample-specific image data, to determine a predicted probe intensity value based on at least one of an input of the probe sequence or the one or more probe features. For example, the predicted probe intensity may be the predicted total signal intensity R_{predicted} of a probe signal. Training data may be held out from the sample-specific image data that is received from the genotyping device or components thereof. When the observed probe intensity value is a raw probe intensity value R (e.g., Raw R), the predicted total signal intensity R_{predicted} may be a predicted raw probe intensity value Raw R_{predicted}. When the observed probe intensity value is a normalized probe intensity value (e.g., Norm R, ENorm R, or LRR), the predicted total signal intensity R_{predicted} may be the same normalized probe intensity value (e.g., Norm R_{predicted}, ENorm R_{predicted}, or LR R_{predicted}).

Each of the machine learning models (e.g., linear regression, random forest, or neural network) may be trained as described herein to optimize the predicted total signal intensity R_{predicted}. As the machine learning models may each be trained using sample-specific image data, the machine learning models may make sample-specific predictions to optimize the predicted total signal intensity R_{predicted} for a given sample.

The training may be reduced by using the set of features as input or a subset of the features. In an example, a single feature of TC may be used as input to reduce training time and processing. As the machine-learning model is trained using sample-specific image data, the machine learning model may be retrained for each new sample or data set.

The trained machine learning model may be implemented (e.g., during production) to generate the predicted total signal intensity R_{predicted} of a probe signal and the predicted total signal intensity R_{predicted} of a probe signal may be used in various applications. FIG. 17B is a flowchart depicting an example procedure 1720 for predicting the total signal intensity R_{predicted} of a probe signal and applying the predicted total signal intensity R_{predicted}. The one or more portions of the procedure 1720 may be performed by one or more computing devices. One or more portions of the procedure 1720 may be stored in memory as computer-readable or machine-readable instructions that may be executed by a processor of the one or more computing devices. Though portions of the procedure 1720 may be described herein as being performed by a single computing device, the procedure 1720, or portions thereof, may be distributed across multiple devices, such as a client computing device, a genotyping device, and/or one or more server computing devices.

The procedure 1720 may begin at 1722. As shown in FIG. 17B, at 1722 a probe sequence and/or probe features that effect the total probe intensity values of probe samples may be received by the machine learning model. The probe sequence and/or probe features may be received as input data at the machine learning model. As described herein, the linear regression model, the random forest model, and/or the neural network may receive a set of probe features or a subset of probe features (e.g., k-mer features). The neural network may also, or alternatively receive a probe sequence (e.g., 50 bp) as input. The neural network may be a hybrid neural network capable of receiving the probe sequence and a predefined set of probe features.

In response to receiving the probe sequence and/or the probe features at 1722, the machine learning model may predict the total signal intensity R_{predicted} at 1724. As described herein, the machine learning model may be trained to predict a raw probe intensity value R (e.g., Raw R_{predicted}) or a normalized probe intensity value (e.g., Norm R_{predicted}, ENorm R_{predicted}, or LRR_{predicted}).

At 1726, the predicted total signal intensity values R_{predicted} may be applied. Different predicted total signal intensity values R_{predicted} may have different applications. For example, when a machine learning model has been trained to predict a raw probe intensity value R (e.g., Raw R) for the predicted total signal intensity value R_{predicted}, the predicted raw probe intensity value Raw R_{predicted} may be used instead of an estimated total signal intensity value that may rely on external controls or reference datasets. For example, the predicted raw probe intensity value Raw R_{predicted} may be used for background and gradient removal. The predicted raw probe intensity value Raw R_{predicted} may be a sample-specific value that may predict the expected intensity level in a region of the image data that is received for a particular sample. The computing device may then perform image processing to subtract out the background or gradient based on the predicted raw probe intensity value Raw R_{predicted}. This more accurate prediction may allow for a better estimate of the true signal for genotype calling. As the predicted raw probe intensity value Raw R_{predicted} may be a sample-specific value, the model may be re-trained for each sample or data set.

When a machine learning model has been trained to predict the total signal intensity value R_{predicted} using Norm R (e.g., Norm R_{predicted}) or LRR (e.g., LRR_{predicted}) the predicted total signal intensity value R_{predicted} may be used instead of an estimated total signal intensity value that may rely on external controls or reference datasets. For example, the predicted total signal intensity value R_{predicted} may be used for additional normalization of the probe signal that is received in the raw signal data. The computing device may perform a partial normalization of the raw signal data to generate Norm R and/or LRR, which may be used to train the machine learning model, as described herein. After the predicted total signal intensity value R_{predicted} (e.g., Norm R_{predicted} or LRR_{predicted}) is determined from the machine learning model, the predicted total signal intensity value R_{predicted} (e.g., Norm R_{predicted} or LRR_{predicted}) may replace the Norm R value or the LRR value to improve the normalized signal. As one example illustrated in Equation 3 herein, the LRR value that may be used for CNV calling may be calculated based on an expected normalized signal intensity value R_{expected}. This expected normalized signal intensity value R_{expected} may be an external control or a dataset that is not based on sample-specific data and may be replaced with the predicted normalized signal intensity Norm R_{predicted} that is based on sample-specific data. As Norm R_{predicted} and LRR_{predicted} may be sample-specific values, the machine learning model may be re-trained for each sample or data set. When performing CNV calling, the LRR value or other normalized value may be compared with the LRR values or other normalized values from the samples with known copy numbers.

In another example, a normalized consensus model may be used to test or predict a quality level of the design of a probe and whether it will accurately target the genome. The normalized consensus model may be implemented using ENorm or R Norm values. One data point for determining a quality level of the design may be the total intensity of the probe. The machine learning model may be used to predict the total signal intensity value R_{predicted}, which may be used as a metric of the quality level of the probe design. As it may be expensive to test each probe design, the machine learning model may use pretrained models without being re-trained for each probe design and may still be used in this application. However, the machine learning model may also be re-trained for different probe designs.

FIG. 18 is a block diagram illustrating an example computing device 1800. One or more computing devices such as the computing device 1800 may implement one or more features for developing, training, or using the machine learning model described herein and/or one or more applications of the predicted total signal intensity value R_{predicted} that may be predicted by the machine learning model. For example, the computing device 1800 may comprise one or more of the genotyping device 111, the computing devices 114a, and/or the computing devices 114b shown in FIG. 1. As shown by FIG. 18, the computing device 1800 may comprise a processor 1802, a memory 1804, a storage device 1806, an I/O interface 1808, and a communication interface 1810, which may be communicatively coupled by way of a communication infrastructure 1812. In some examples, such as when the computing device 1800 comprises a genotyping device, the computing device 1800 may include a local imaging subsystem comprising imaging components 1814. The imaging components may include optical imaging components and/or digital imaging components. The optical imaging components may include a light source (e.g., lasers, light emitting diodes (LEDs)) tuned to wavelengths of light that induce excitation in a sample; one or more optical instruments, such as cameras, lenses, sensors, detect and image signals emitted through induced excitation, and one or more processors for developing composite images from signals detected. It should be appreciated that the computing device 1800 may include fewer or more components than those shown in FIG. 18.

The processor 1802 may include hardware for executing instructions, such as those making up a computer program. In examples, to execute instructions for dynamically modifying workflows, the processor 1802 may retrieve (or fetch) the instructions from an internal register, an internal cache, the memory 1804, or the storage device 1806 and decode and execute the instructions. The memory 1804 may be a volatile or non-volatile memory used for storing data, metadata, computer-readable or machine-readable instructions, and/or programs for execution by the processor(s) for operating as described herein. The storage device 1806 may include storage, such as a hard disk, flash disk drive, or other digital storage device, for storing data or instructions for performing the methods described herein.

The I/O interface 1808 may allow a user to provide input to, receive output from, and/or otherwise transfer data to and receive data from the computing device 1800. The I/O interface 1808 may include a mouse, a keypad or a keyboard, a touch screen, a camera, an optical scanner, network interface, modem, other known I/O devices or a combination of such I/O interfaces. The I/O interface 1808 may include one or more devices for presenting output to a user, including, but not limited to, a graphics engine, a display (e.g., a display screen), one or more output drivers (e.g., display drivers), one or more audio speakers, and one or more audio drivers. The I/O interface 1808 may be configured to provide graphical data to a display for presentation to a user. The graphical data may be representative of one or more graphical user interfaces and/or any other graphical content.

The communication interface 1810 may include hardware, software, or both. In any event, the communication interface 1810 may provide one or more interfaces for communication (such as, for example, packet-based communication) between the computing device 1800 and one or more other computing devices or networks. The communication may be a wired or wireless communication. As an example, and not by way of limitation, the communication interface 1810 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1810 may facilitate communications with various types of wired or wireless networks. The communication interface 1810 may also facilitate communications using various communication protocols. The communication infrastructure 1812 may also include hardware, software, or both that couples components of the computing device 1800 to each other. For example, the communication interface 1810 may use one or more networks and/or protocols to enable a plurality of computing devices connected by a particular infrastructure to communicate with each other to perform one or more aspects of the processes described herein. To illustrate, the sequencing process may allow a plurality of devices (e.g., a client device, sequencing device, and server device(s)) to exchange information such as sequencing data and error notifications.

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure.

What is claimed is:

1. A computer-implemented method comprising:
receiving sample-specific image data, wherein the sample-specific image data comprises a signal associated with a sample for a probe in a microarray relating to a single individual;
identifying an observed probe intensity value for the sample based on the sample-specific image data;
identifying at least one of a probe sequence or one or more probe features effecting a total probe intensity value for the sample; and
training, using training data derived from the sample-specific image data, a machine learning model to determine a predicted probe intensity value based on an input of the at least one of the probe sequence or the one or more probe features, wherein the predicted probe intensity value is a predicted total signal intensity of the signal associated with the sample for the probe, and wherein the training data is derived from the same sample specific image data as testing data that may be separated for testing the trained machine learning model.

2. The computer-implemented method of claim 1, wherein the sample-specific image data comprises a raw x signal having a first intensity value of a first colored signal that represents a fluorescent label for a genotype A, and wherein the sample-specific image data comprises a raw y signal having a second intensity value of a second colored signal that represents a fluorescent label for a genotype B.

3. The computer-implemented method of claim 2, wherein the observed probe intensity value is a raw probe intensity value or a normalized probe intensity value, and wherein the predicted probe intensity value is a predicted raw probe intensity value or a predicted normalized probe intensity value.

4. (canceled)

5. (canceled)

6. (canceled)

7. The computer-implemented method of claim 1, wherein the machine learning model is a linear regression model or a random forest model, and wherein the input of the one or more probe features comprise k-mer features of the probe.

8. The computer-implemented method of claim 1, wherein the machine learning model is a random forest model or a neural network, and wherein the input of the one or more probe features comprises an entire predefined set of probe features.

9. The computer-implemented method of claim 1, wherein the machine learning model is a neural network, and wherein the input comprises the probe sequence, and wherein the probe sequence is a 50 bp probe sequence.

10. The computer-implemented method of claim 9, wherein the neural network is a hybrid neural network comprising a convolutional portion and a fully-connected feed forward portion, and wherein the input comprises the probe sequence for the convolutional portion and the one or more probe features for the fully-connected feed forward portion.

11. The computer-implemented method of claim 1, wherein the one or more probe features comprise at least one of a primer melting temperature (TM) under one or more salt concentrations or a GC content.

12. The computer-implemented method of claim 1, wherein the sample-specific image data is received from a genotyping device, wherein the microarray comprises a BeadArray.

13. (canceled)

14. (canceled)

15. A computer-readable storage medium having instructions stored thereon that, when executed by a processor, cause the processor to:
receive sample-specific image data, wherein the sample-specific image data comprises a signal associated with a sample for a probe in a microarray relating to a single individual;
identify an observed probe intensity value for the sample based on the sample-specific image data;
identify at least one of a probe sequence or one or more probe features effecting a total probe intensity value for the sample; and
train, using training data derived from the sample-specific image data, a machine learning model to determine a predicted probe intensity value based on an input of the at least one of the probe sequence or the one or more probe features, wherein the predicted probe intensity value is a predicted total signal intensity of the signal associated with the sample for the probe, and wherein the training data is derived from the same sample specific image data as testing data that may be separated for testing the trained machine learning model.

16. The computer-readable storage medium of claim 15, wherein the sample-specific image data comprises a raw x signal having a first intensity value of a first colored signal that represents a fluorescent label for a genotype A, and wherein the sample-specific image data comprises a raw y signal having a second intensity value of a second colored signal that represents a fluorescent label for a genotype B.

17. The computer-readable storage medium of claim 16, wherein the observed probe intensity value is a raw probe intensity value or a normalized probe intensity value, and wherein the predicted probe intensity value is a predicted raw probe intensity value gr a predicted normalized probe intensity value.

18. (canceled)

19. (canceled)

20. (canceled)

21. The computer-readable storage medium of claim 15, wherein the machine learning model is a linear regression model or a random forest model, and wherein the input of the one or more probe features comprise k-mer features of the probe.

22. The computer-readable storage medium of claim 15, wherein the machine learning model is a random forest model or a neural network, and wherein the input of the one or more probe features comprises an entire predefined set of probe features.

23. The computer-readable storage medium of claim 15, wherein the machine learning model is a neural network, and wherein the input comprises the probe sequence, and wherein the probe sequence is a 50 bp probe sequence.

24. The computer-readable storage medium of claim 23, wherein the neural network is a hybrid neural network comprising a convolutional portion and a fully-connected feed forward portion, and wherein the input comprises the probe sequence for the convolutional portion and the one or more probe features for the fully-connected feed forward portion.

25. The computer-readable storage medium of claim 15, wherein the one or more probe features comprise at least one of a primer melting temperature (TM) under one or more salt concentrations or a GC content.

26. The computer-readable storage medium of claim 15, wherein the sample-specific image data is received from a genotyping device, wherein the microarray comprises a BeadArray.

27. (canceled)

28. (canceled)

29. A system comprising:
an imaging system configured to capture image data and generate sample specific image data; and
at least one processor configured to:
receive sample-specific image data, wherein the sample-specific image data comprises a signal associated with a sample for a probe in a microarray relating to a single individual;
identify an observed probe intensity value for the sample based on the sample-specific image data;
identify at least one of a probe sequence or one or more probe features effecting a total probe intensity value for the sample; and
train, using training data derived from the sample-specific image data, a machine learning model to determine a predicted probe intensity value based on an input of the at least one of the probe sequence or the one or more probe features, wherein the predicted probe intensity value is a predicted total signal intensity of the signal associated with the sample for the probe, and wherein the training data is derived from the same sample specific image data as testing data that may be separated for testing the trained machine learning model.

30. The system of claim 29, wherein the sample-specific image data comprises a raw x signal having a first intensity value of a first colored signal that represents a fluorescent label for a genotype A, and wherein the sample-specific image data comprises a raw y signal having a second intensity value of a second colored signal that represents a fluorescent label for a genotype B.

31. The system of claim 30, wherein the observed probe intensity value is a raw probe intensity value or a normalized probe intensity value, and wherein the predicted probe intensity value is a predicted raw probe intensity value or a predicted normalized probe intensity value.

32. (canceled)

33. (canceled)

34. (canceled)

35. The system of claim 29, wherein the machine learning model is a linear regression model or a random forest model, and wherein the input of the one or more probe features comprise k-mer features of the probe.

36. The system of claim 29, wherein the machine learning model is a random forest model or a neural network, and wherein the input of the one or more probe features an entire predefined set of probe features.

37. The system of claim 29, wherein the machine learning model is a neural network, and wherein the input comprises the probe sequence, and wherein the probe sequence is a 50 bp probe sequence.

38. The system of claim 37, wherein the neural network is a hybrid neural network comprising a convolutional portion and a fully-connected feed forward portion, and wherein the input comprises the probe sequence for the convolutional portion and the one or more probe features for the fully-connected feed forward portion.

39. The system of claim 29, wherein the one or more probe features comprise at least one of a primer melting temperature (TM) under one or more salt concentrations or a GC content.

40. The system of claim 29, wherein the imaging device resides on a genotyping device, wherein the at least one processor resides on a separate computing device, and wherein the microarray comprises a BeadArray.

41. (canceled)

42. (canceled)

43. The system of claim 29, wherein the imaging system is a local imaging subsystem of a computing device that also comprises the at least one processor.

44-87. (canceled)
